(19) United States
(12) Patent Application Publication
(10) Pub. No.: US 2026/0132081 A1
(43) Pub. Date: May 14, 2026

(54) MULTIFUNCTIONAL DAMAGE RESPONSIVE POLYMERIC FIBER

(71) Applicant: Drexel University, Philadelphia, PA (US)

(72) Inventors: Yaghoob Farnam, Philadelphia, PA (US); Caroline L Schauer, Hulmeville, PA (US); Cristopher Michael Sales, Philadeliphia, PA (US); Ahmad Raeisi Najafi, Chester Springs, PA (US)

(73) Assignee: Drexel University, Philadelphia, PA (US)

(21) Appl. No.: 19/118,400

(22) PCT Filed: Oct. 17, 2023
(86) PCT No.: PCT/US2023/077046

§ 371 Date: Apr. 4, 2025

Related U.S. Application Data
(60) Provisional application No. 63/379,970, filed on Oct. 18, 2022.
(60) Provisional application No. 63/482,994, filed on Feb. 2, 2023.
(60) Provisional application No. 63/512,194, filed on Jul. 6, 2023.

(51) Int. Cl. C04B 20/12; C04B 16/06; C04B 20/10; C04B 103/00; C12N 11/04; C12N 11/082; C12N 11/084; C12N 11/096; C12N 11/12; D06B 3/02; D06B 21/00; D06M 15/07; D06M 15/13; D06M 15/233; D06M 15/256; D06M 15/263; D06M 15/356; D06M 15/507; D06M 15/55; D06M 16/00; D06M 23/10; D06M 101/24; D06M 101/32

(52) U.S. Cl. CPC C04B 20/12; C04B 16/0641; C04B 16/0683; C04B 20/1033; C04B 20/1037; C04B 20/1048; C12N 11/04; C12N 11/082; C12N 11/084; C12N 11/096; C12N 11/12; D06B 3/02; D06B 21/00; D06M 15/07; D06M 15/13; D06M 15/233; D06M 15/256; D06M 15/263; D06M 15/3562; D06M 15/507; D06M 15/55; D06M 16/003; D06M 23/10; C04B 2103/0001; D06M 2101/24; D06M 2101/32

(57) ABSTRACT

The present application relates to a multifunctional-damage responsive biofiber and methods of forming thereof, comprising: a core comprising a polymeric fiber; a crosslinked endospore loaded hydrogel layer coating the core, wherein the hydrogel layer is formed with: a solution comprising endospores, one or more anionic polymers, and one or more crosslinking agents; and a (co)polymer shell encapsulating the hydrogel layer, wherein the shell has one or more layers which may be the same or different, and each of the one or more layers is formed with a (co)polymer selected from the group consisting of nitrocellulose (NITR), epoxy Resin (ER), polymethylmethacrylate (PMMA), polyvinylidene fluoride (PVDF), cyanoacrylate adhesive (CYA), polystyrene (PS), polylactic acid (PLA), and combinations thereof. This application also relates to methods for self-healing a matrix material including impregnating a matrix material with the multi-functional damage-responsive biofiber.

### CROSS REFERENCE TO RELATED APPLICATIONS

This application is a 371 continuation of International application no. PCT/US2023/077046, filed on Oct. 17, 2023, which, in turn, claims the benefit of U.S. Provisional Application No. 63/379,970 filed on Oct. 18, 2022, U.S. Provisional Application No. 63/482,994 filed on Feb. 2, 2023, and U.S. Provisional Application No. 63/512,194 filed on Jul. 6, 2023, the disclosures of which are hereby incorporated by reference in their entirety as if set forth fully herein.

### STATEMENT OF GOVERNMENT INTEREST

This invention was made with government support under contract no. 2029555 awarded by the National Science Foundation. The Government has certain rights in the invention.

### BACKGROUND OF THE INVENTION

Responsive fibers are of interest due to their potential use in self-healing, sensing, drug delivery, and microcrack identification in materials [1,2]. Current methods for making responsive fibers primarily involve stimuli responsive materials. These stimuli responsive materials react to an external stimulus such as temperature, pH, light, an electric field, a magnetic field or pressure by undergoing a physical change. Common approaches for damage responsive materials is to incorporate microcapsules or hollow fibers which are then loaded with molecules, such as fluorogenic materials. When the material is damaged, the microcapsules or hollow fibers release their contents, for example, fluorogenic materials that can be used to identify damaged areas [1,2].

Although these damage responsive microcapsules or hollow fibers may achieve the self-healing goal, they do not provide any mechanical strength to the polymer material. Self-healing is the intrinsic capability of a material to repair a physical degradation without external intervention and either, partially or fully recover to its pre-damage state [1-4]. Natural organisms demonstrate the ability to self-heal when non-catastrophic damages occur due to intrinsic complications or/and extrinsic menaces [5-8]. The self-healing functionality in living organisms grants extended longevity and adaptation to environmental changes [9, 10]. Wound healing, for instance, is one of the common self-healing processes in biological systems. The performance of engineering materials is dominated by irreversible chemical/physical degradation processes which leads to thermodynamic, chemical, and mechanical instability of materials [11-14]. The degradation mechanism involves a combination of internal and external factors, including alterations in microstructure and composition, deformation over time, exposure to corrosive substances, and challenging environmental conditions [15]. The execution of strategies aimed at preventing or mitigating degradation can incur significant costs, consume considerable time, and pose practical challenges [16].

Self-healing capability could provide shelf-life extension and enhanced mechanical integrity to engineering materials. Researchers have shown growing interest in nature-inspired self-healing functionality to integrate self-healing properties into engineering materials [17, 18], which can prolong the service-life of materials, reduce the materials inefficiency, and have desirable economic attributes [19]. Moreover, self-healing materials are suggested to provide sustained performance reliability, especially in areas with limited accessibility [20]. Considering that different living organisms have distinctive healing mechanisms [21] it is hypothesized that the healing process and its delivery strategy need to be tailored for each engineering material [20]. In the realm of self-healing materials, a biomimetic engineered materials are those featuring self-healing features mainly through systematic transport of self-healing agents [22]. Self-healing mechanisms have been integrated into various materials such as metals, alloys, ceramics, polymers, and composites [23].

An innovative autonomous self-healing mechanism used in quasi-brittle engineering materials is to utilize microbial-induced calcium carbonate precipitation (MICCP) process [24]. This technique is performed through the reaction of carbonate, a common metabolic end-product of many microorganisms, with calcium ions to form calcium carbonate. The harsh conditions in concrete, e.g., high pressure, temperature, alkaline condition, and unavailability of sufficient oxygen, can adversely affect microbial activity and survivability [25-27]. Since endospores of bacteria, metabolically inactive form of vegetative cells, can remain dormant for prolonged periods with high survivability rate in extreme conditions, this bacterial phenotype is often favored for MICCP [28]. In the presence of carbon and nutrient sources, endospores can be induced to from its dormant state (endospores) to become vegetative cells in a process initiated by germination [29]. In addition to using endospores, several delivery methods such as encapsulation and creation of vascular network, have been applied to protect bio-agents to deliver and integrate autonomous self-healing functionality into matrices [30-36].

The encapsulation of self-healing agents in different carries have been proposed in the art to protect bacteria and self-healing agents in quasi-brittle composites from extreme environments and harsh manufacturing processes [30]. When cracks form in quasi-brittle materials, the carrier ruptures along the crack path, leading to the release of healing agents into the crack due to capillary pressure and gravitational forces [37]. Either organic or inorganic materials can be used to synthesize healing agent's carrier capsule [38], including polyurethane, polyurethane, ethylene cellulose, poly styrene-divinylbenzene, urea-formaldehyde, phenol-formaldehyde resin, bio-hydrogel, alginate, colloidal silica, silica gel, sodium silicate, melamine formaldehyde, bentonite nano/micro particles, graphite nano-plate [39-42].

One of the major challenges in autonomous encapsulation method is to control self-activation of healing agents (i.e., damage-responsiveness or induction of MICCP activity of the spores) to coincide with crack occurrences [43]. To address this issue, several studies have investigated the development of shell/core capsules that have a brittle strain-responsive shell material that breaks upon cracking [44]. Jiang et al. manufactured a composite shell structure using melamine phenolic resin with modified sodium alginate [42]. The particle size, thermal stability, and coating ratio of micro-capsules were tuned by sodium alginate properties such as adhesion and degradability [42]. Melamine phenolic resin provided brittleness and hardness to the capsule shell. For the capsule core, two-component epoxy resin was used as healing agent [42]. Despite the significant advantages of autonomous encapsulation technologies, these techniques have not been widely mass-produced due to their complicated preparation process, resulting in an uneven distribution, lower mechanical properties, and expensive manufacturing process/material [45, 46].

Several researchers also investigated the feasibility of embedding damage-responsive brittle hollow tubes, or creating vascular network in composite materials, to incorporate chemical/bacterial healing agents into them [47-49]. These tubes and vascular network provide large void volumes to incorporate higher amounts of healing agents. In addition, the vascular network creation can be controlled in the manufacturing process, preventing the dispersion randomness of the spherical capsules [48]. A novel approach to create vascular network in the composite is using 3D printing approach [35, 36, 50]. The three-dimensional vascular structures can be created with any complex geometry based on the target application. In terms of autonomous vascular self-healing, brittle/removeable materials are used to facilitate initiation of self-healing mechanism. The 3D channel network provides transportation of healing agents into the matrix. Furthermore, several studies have investigated the influence of vascular network on mechanical/fracture properties of the concrete as well as the self-healing efficiency. Using incompatible materials, e.g., silicone, polyvinyl chloride, and polyethylene terephthalate, which possess different elastic modulus, with concrete may cause delamination, resulting in lower autonomous healing efficiency [35, 36]. In a study done by Wan et al., they designed an octet-structure lattice made of acrylonitrile butadiene styrene (ABS) for creating the vascular network to deliver the healing agent to the crack regions [35]. Although imitating self-healing in materials has introduced an era of novel smart materials, the field is in the infancy. Designing self-healing processes in materials by well-established synthetic paths is challenging [51].

Accordingly, there are various aspects of self-healing that are not thoroughly covered in the current state of the literature. One of the highly important aspects is to incorporate self-healing robustness in terms of featuring damage-responsiveness and controlled crack propagation. A major challenge in autonomous self-healing is to control crack growth while healing takes place, similar to skin biological tissues in nature. Furthermore, the existing literature lacks extensive understanding regarding the enhancement of the survival and long-term effectiveness of bioinspired or bio-based healing agents. Hence, investigating the feasibility of developing a bacterial-based self-healing system with controlled crack growth and damage-responsive capabilities is of paramount significance.

### SUMMARY OF THE INVENTION

The present invention may be description by the following sentences.
1. In a first aspect, the present invention relates to a multifunctional-damage responsive biofiber including:
   a core comprising a polymeric fiber comprising one or more polymers selected from the group consisting of polyester, polyethylene, polypropylene, polyvinyl alcohol, polyamides, aramid, polyacrylonitrile, cellulose, polyurethane, and combinations thereof;
   a crosslinked endospore loaded hydrogel layer coating the core, wherein the hydrogel layer is formed with:
      a solution comprising endospores and one or more anionic polymers selected from the group consisting of polysaccharides, hyaluronic acid, colominic acid, polysalic acid, chondroitin, queratane, dextrans, heparin, carrageenan, furcelerans, alginates, agar agar, glucomannan, gums, pectins, cellulose, starches, sorbitan esters, and combinations thereof, and
      one or more crosslinking agents; and
   a (co)polymer shell encapsulating the hydrogel layer, wherein the shell has one or more layers which may be the same or different, and each of the one or more layers is formed with a (co)polymer selected from the group consisting of nitrocellulose (NITR), epoxy Resin (ER), polymethylmethacrylate (PMMA), polyvinylidene fluoride (PVDF), cyanoacrylate adhesive (CYA), polystyrene (PS), polylactic acid (PLA), and combinations thereof.
2. The biofiber of sentence 1, wherein the one or more anionic polymers may include sodium alginate.
3. The biofiber of any one of sentences 1-2, wherein the one or more crosslinking agents may be a cationic crosslinking agent selected from the group consisting of calcium chloride, calcium acetate, and combinations thereof.
4. The biofiber of any one of sentences 1-3, wherein the shell may be formed from a copolymer.
5. The biofiber of sentence 4, wherein the copolymer may include a combination of two of the (co)polymers in a weight ratio of from about 0.25:1 to 1:0.25, or about 1:1.
6. The biofiber of any one of sentences 1-5, wherein the shell may be formed from two or more layers, or three or more layers, or four or more layers, or five layers.
7. The biofiber of any one of sentences 1-6, wherein the hydrogel layer may have a thickness of from about 0.5% to about 20% when compared to the thickness of the core.
8. The biofiber of any one of sentences 1-7, wherein the polymeric fiber may have a fiber length of from about 10 mm to about 100 mm, or from about 10 mm to about 70 mm, or from about 10 mm to about 60 mm.
9. The biofiber of any one of sentences 1-8, wherein the polymeric fiber may have a ratio of a fiber length to a fiber diameter of from about 20 to 80 or from about 20 to about 60.
10. The biofiber of any one of sentences 1-9, wherein the hydrogel layer may have a swelling ratio of from about 0 to 10, or from about 0.2 to 8, after 15 minutes of exposure to an aqueous solution, wherein the swelling ratio is determined by Equation 4:

$${s}_{r} = \frac{{W}_{s} - {W}_{d}}{{W}_{d}}$$

wherein sᵣ is the swelling ratio, wₛ is the weight of the hydrogel layer after exposure to the aqueous solution, and w_{d} is the weight of the hydrogel layer prior to exposure to the aqueous solution.
11. The biofiber of any one of sentences 1-10, wherein the hydrogel layer may be configured to release the endospores upon fracture of the shell layer.
12. The biofiber of any one of sentences 1-11, wherein the biofiber may be configured to have a function selected from the group consisting of self-healing, sensing, drug delivery, and microcrack identification in materials.
13. The biofiber of any one of claims 1-12, wherein the one or more anionic polymers in the solution may have a concentration of from about 1% w/v to about 20% w/v, or from about 2% w/v to about 15% w/v, based on the weight of the one or more anionic polymers in a solvent.
14. In a second aspect, the present invention may relate to a method of forming the multi-functional damage-responsive biofiber of any one of sentences 1-13, comprising steps of:
a) coating the core with the solution comprising the one or more anionic polymers and endospores to form a coated core;
b) crosslinking the coated core formed in step a) with the crosslinking agent to form a crosslinked hydrogel layer coated core;
c) encapsulating the crosslinked hydrogel layer coated core with the (co)polymer to form a polymer shell having one or more layers on the exterior in order to form the multi-functional biofiber.
15. The method of sentence 14, may further comprise a step of drying the crosslinked hydrogel layer coated core.
16. The method of any one of sentences 14-15, wherein the coating of the core in step a) may be performed with a reel-to-reel system, at a speed of 10 rot/sec to 100 rot/sec or, more preferably, 50 rot/sec to 100 rot/sec.
17. The method of any one of sentences 14-15, wherein step a) may be carried out by soaking the core in the solution, and/or wherein step b) may be carried out by soaking the product of step a) in a bath comprising the crosslinking agent and crosslinking the crosslinking agent to form the crosslinked hydrogel layer coated core, and/or wherein step c) may be carried out by soaking the crosslinked hydrogel layer coated core in a bath of the (co)polymer to encapsulate the crosslinked hydrogel layer coated core thereby forming the multi-functional biofiber.
18. In a third aspect, the present invention may relate to a method for self-healing a matrix material, comprising impregnating the matrix with the multi-functional damage-responsive biofiber of any one of claims 1-13 to form an impregnated matrix, wherein the multi-functional damage-responsive biofiber releases the endospore hydrogel layer upon fracture of the shell, corresponding to a fracture of the matrix to induce self-healing.
19. The method of sentence 18, wherein the matrix, the core, and the shell of the multi-functional damage-responsive biofiber are defined by Equation A:

$$0.8 \leq \frac{{G}_{shell}}{{G}_{matrix}} < 1. and \frac{{G}_{shell}}{{G}_{core}} \leq 1.$$

wherein Gₛₕₑₗₗ is defined as a fracture energy release rate of the polymer shell, Gₘₐₜᵣᵢₓ is defined as the fracture energy release rate of the impregnated matrix, and G_{core} is defined as the fracture energy release rate of the core.
20. The method of any one of sentences 18-19, wherein the matrix material is not cement.

Additional details and advantages of the disclosure will be set forth in part in the description which follows, and/or may be learned by practice of the disclosure. The details and advantages of the disclosure may be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

FIG. 1 shows a schematic illustration of BioFiber self-healing stages in a quasi-brittle composite shown in grey color.

FIG. 2A shows hydrogel coating thickness of polyester versus concentration of sodium-alginate solution.

FIG. 2B shows hydrogel coating thickness of polyvinyl alcohol versus concentration of sodium-alginate solution (Na-Alg). The statistic shown in the plot of FIGS. 2A-2B is based on 30 replicates for each set of samples.

FIG. 3 shows in image a) a scanning electron microscopy (SEM) images of polyester core-fiber without hydrogel coating. Image b) shows SEM images of polyvinyl alcohol core-fiber without hydrogel coating.

FIG. 4. shows in image a) a SEM image of the core-fiber with hydrogel coating of polyester with 2% w/v Na-Alg. Image b) shows SEM images of the core-fiber with hydrogel coating for polyvinyl alcohol with 2% w/v Na-Alg.

FIG. 5A shows normalized weight of the hydrogel coating (W_{Hgel}) (mg/mg/cm) of a polyester core fiber. The normalized weight of the hydrogel coating (W_{Hgel}) may be calculated by Equation 2.

FIG. 5B shows normalized weight of the hydrogel coating (W_{Hgel})(mg/mg/cm) of a polyvinyl alcohol core fiber. The normalized weight of the hydrogel coating W_{Hgel}) may be calculated by Equation 2. The information shown in these plots for FIGS. 5A-5B is based on 30 replicates for each set of samples.

FIG. 6A shows the hydrogel swelling ratio (g/g) for polyester when exposed to deionized (DI) water for 15 min for hydrogel layers formed with different concentrations of Na-Alg.

FIG. 6B shows the hydrogel swelling ratio g/g for polyvinyl alcohol when exposed to the deionized (DI) water for 15 min for hydrogel layers formed with different concentrations of Na-Alg. The y-axis in FIGS. 6A-6B indicates the weight of solution, in grams, absorbed per weight of the BioFiber as described in Eq. 4. The information shown in these plots for FIGS. 6A-6B is based on 10 replicates for each set of samples.

FIG. 7A shows the hydrogel swelling ratio for a polyester for 8 w/v Na-Alg crosslinked with calcium acetate (CA), versus time of exposure to DI water.

FIG. 7B shows the hydrogel swelling ratio for a polyvinyl alcohol for 8 w/v Na-Alg crosslinked with calcium acetate (CA), versus time of exposure to DI water. The information shown in the plots of FIGS. 7A-7B is based on 10 replicates for each set of samples.

FIG. 8A shows the hydrogel swelling ratio for a polyester with 8 w/v Na-Alg crosslinked with CA versus pH of the solution, with 15 minutes of exposure.

FIG. 8B shows the hydrogel swelling ratio for a polyvinyl alcohol with 8 w/v Na-Alg crosslinked with CA versus pH of the solution, with 15 minutes of exposure. The information shown in the plots of FIGS. 8A-8B is based on 10 replicates for each set of samples.

FIG. 9 shows examples of shell coating materials on hydrogel coated core-fibers.

FIG. 10 shows average shell coating thicknesses on PES using polylactic acid:polystyrene (PLA:PS) (1:1 wt. %) at varying concentrations of the copolymer in the solvent. The information shown in this plot is based on 10 replicates for each set of samples.

FIG. 11 shows average shell coating thicknesses on PVA using PLA:PS (1:1 wt. %) at varying concentrations of the copolymer in the solvent. The information shown in this plot is based on 10 replicates for each set of samples.

FIG. 12A shows the shell coating thicknesses using PLA:PS (1:1 wt. %) at varying concentrations of the copolymer in the solvent for the PES of FIG. 10. The information shown in this plot is based on 10 replicates for each set of samples.

FIG. 12B shows the shell coating thicknesses using PLA:PS (1:1 wt. %) at varying concentrations of the copolymer in the solvent for polyvinyl alcohol of FIG. 11. The information shown in this plot is based on 10 replicates for each set of samples.

FIG. 13A shows the percentage of added thickness when one layer of hydrogel/shell was applied to the core-fiber for polyester.

FIG. 13B shows the percentage of added thickness when one layer of hydrogel/shell was applied to the core-fiber for polyvinyl alcohol.

FIG. 14 shows SEM images of the core-fiber coated with hydrogel and shell (PLA:PS 6 w/v, 1-layer) at 2.00 magnification (kX), in image (a), and at 4.40 kX, in image (b).

FIG. 15 shows SEM images of the core-fiber coated with hydrogel and shell (PLA:PS 18 w/v, 1-layer) at 2.00 kX, in image (a), and at 4.40 kX, in image (b).

FIG. 16A shows BioFiber survivability test results against casting mechanical forces for polyester.

FIG. 16B shows BioFiber survivability tests results against casting mechanical forces for polyvinyl alcohol.

In FIG. 16A-16B, the survivability is defined as the number of BioFibers that remained intact after a simulated casting process with respect to the total number of BioFibers used.

FIG. 17A shows a set of representative thermogravimetric analysis results for BioFiber made of polyester.

FIG. 17B shows a set of representative thermogravimetric analysis results for BioFiber made of polyvinyl alcohol.

FIG. 18A shows the calcium carbonate quantification for intact and fractured amounts of calcium carbonate, in percentage, normalized to the total weight of the tested samples.

FIG. 18B shows the calcium carbonate quantification for intact and fractured total amounts of calcium carbonate (in mg) precipitated per one BioFiber, wherein the information shown in the this plot is based on five replicates for each set of samples.

FIG. 19 shows SEM images of the fractured BioFiber before Microbial Induced Calcium Carbonate Precipitation (MICCP), in image (a), and after MICCP, in image (b).

FIG. 20 shows in image (a) a BioFiber-reinforced concrete under load. Image (b) shows an example of material property mismatch, wherein the fracture toughness of the shell is significantly less than the fracture toughness of the concrete and the fracture toughness of the shell is significantly less than the fracture toughness of the core. In this example, the polymeric shell breaks prior to the crack reaching the BioFiber. Image (c) shows an example of the desired outcome, wherein the ratio of the fracture release rate (G) of the shell to the G of the concrete is 0.8 and greater up to less than 1.0. In this scenario, the shell breaks once the crack comes into contact with the shell, and the healing agent is released. In addition, the fibers in front of the crack tip, due to higher toughness, will deflect the cracks. Image (d) shows another undesirable outcome, wherein the G of the shell is greater than the G of the concrete and the G of the shell is greater than the G of the core. In this scenario, since the fiber is tougher than the concrete, the fiber does not break, and the healing agent is not released.

FIG. 21 shows a schematic of the geometrical parameters analyzed herein for the BioFibers in the finite element method (FEM).

FIG. 22 shows the crack propagation and toughening mechanism in BioFibers under (a) tension, (b) compression, and (c) three-point bending. In each of the models, l/d (ratio of fiber length to diameter)=40 and t (shell thickness)=0.2 mm, the contour plot exhibits damage parameter, wherein 0 means no damage, and 1 denotes completely damaged. In all the results above, the elastic moduli of the matrix, shell, and fiber were 25000 MPa, 4000 MPa, and 1000 MPa, respectively. The energy release rates were 0.5 N/mm, 0.4 N/mm, and 1 N/mm, and the Poisson's ratios were 0.19, 0.3, and 0.2 of the matrix, shell, and fiber, respectively.

### DETAILED DESCRIPTION

The present invention relates to a multi-functional damage responsive polymeric fiber material which is suitable to act as a delivery system while simultaneously providing mechanical strength.
A simple and cost-effective damage responsive multifunctional fiber has been made.
These multifunctional fibers can be utilized for self-healing polymers or quasi-brittle materials by incorporating self-healing agents into the alginate layer.
The properties of the outer shell can be tuned to the mechanical properties required of the polymeric material such that a fracture of the polymeric material will break the polymer shell and release the self-healing agent under exposure to water.
   Current methods existing in the art do not simultaneously provide crack bridging and autonomous healing.

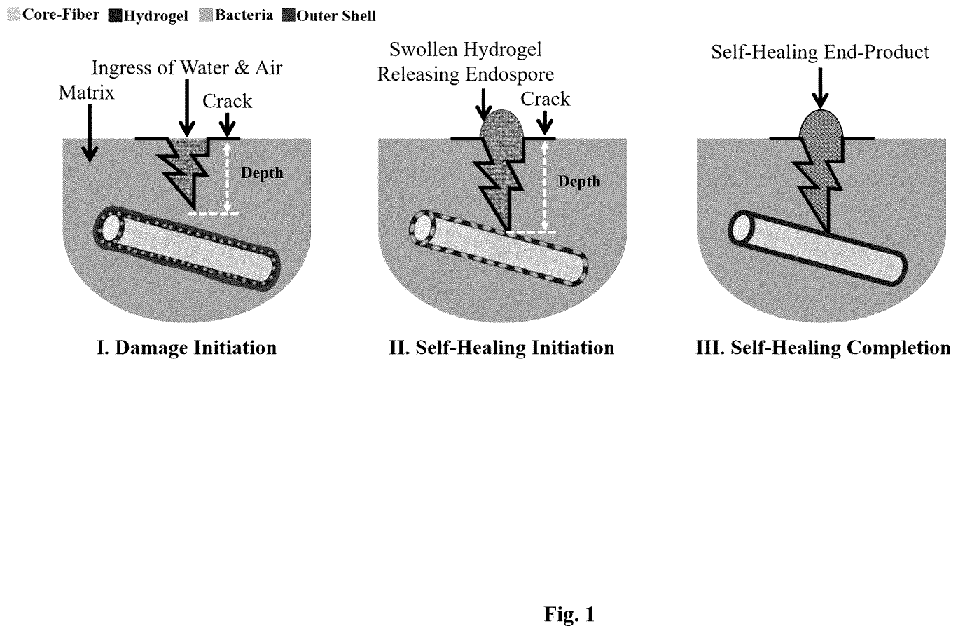

The present invention relates to a novel delivery/activation system to establish a robust autonomic self-healing paradigm by developing multi-functional microbial-based fibers, referred to as “BioFiber” herein, for bio-self-healing agent delivery in quasi-brittle materials. The proposed Bio-Fiber self-healing strategy in quasi-brittle materials is schematically illustrated in FIG. 1. In the first stage, damage initiation, crack opens on the surface of a matrix allowing water and oxygen penetrates through micro-crack from surrounding environment. In the second stage, cracks propagate, breaking the damage-responsive impermeable fiber shell layer, exposing the endospore-laden hydrogel to water and oxygen. The hydrogel swells and disperses the endospore into the crack volume, exposing them to water, oxygen, and nutrients within crack volume. In the third stage, the germinated endospore produces self-healing end-products, i.e., Microbial Induced Calcium Carbonate Precipitation (MICCP), healing the exposed cracks.

The present invention relates to nature-inspired multi-functional polymeric fibers, also referred to as “BioFiber” herein. The BioFiber disclosed herein may be used to deliver bio-self-healing agents into quasi-brittle materials. The BioFibers of the present invention were manufactured using a load-bearing core-fiber, a sheath of endospore-laden hydrogel, and an outer damage-responsive polymeric shell layer. The innovative BioFiber integrates three key functionalities into the quasi-brittle matrix: (i) autonomous bio-self-healing, (ii) crack growth control, and (iii) damage-responsiveness. The hydrogel sheath contained endospores, as bio-agents, to establish microbially-induced calcium carbonate precipitation (MICCP) as a self-healing end-product. The core-fibers provided crack growth control functionality into quasi-brittle engineering materials. Additionally, the outer shell coating integrated a robust damage-responsive self-healing activation strategy in quasi-brittle materials. The examples disclosure herein revealed that a concentration of 8 w/v sodium-alginate crosslinked with calcium acetate provided higher solution uptake capacity required for MICCP. As for the shell, the polymer blend of polystyrene and polylactic acid (1:1 wt. %), with polymer/solvent ratio of 18 w/v-single layer coating, effectively protected BioFibers during a simulated casting process for quasi-brittle materials. Lastly, each BioFiber was able to produce 40-80 mg of calcium carbonate within the first 30 hours of activation.

The effectiveness of the present invention was tested using a variety of materials and manufacturing methods to create the three different layers of the BioFibers based on their targeted functionality: (i) polymeric core-fiber as load-bearing/energy-absorber element, (ii) sheath of endospore-laden alginate hydrogel, and (iii) damage-responsive polymeric outer shell layer. To integrate damage-induced healing mechanism into the BioFiber, a damage-responsive polymeric-based shell coating was developed as the outer layer on the hydrogel coated core-fiber based on the following three criteria have been defined as: (i) desirable coating layer morphology in terms of low coating-to-fiber ratio and uniformity, (ii) impermeability to protect endospore-laden hydrogel from undesired access to moisture/aqueous solution, and (iii) abrasion resistance during blending with a quasi-brittle material.

The present invention relates to multi-functional damage responsive polymeric fibers, methods of making the multi-functional damage responsive polymeric fibers, and methods for self-healing a composite comprising the multi-functional damage responsive polymeric fibers. The present invention demonstrates the following performance capabilities of the innovative multi-functional bacterial-based polymeric fibers: (i) autonomous MICCP self-healing, (ii) crack growth control, and (iii) damage-responsiveness.

### Multi-Functional Damage Responsive Polymeric Material

### Core

The multi-functional damage responsive polymeric fiber of the present invention includes a core. Suitable materials for the core of the multi-functional damage responsive polymeric fiber may include polyester, polyvinyl alcohol, polyethylene, polypropylene, polyamides, aramid, polyacrylonitrile, cellulose, and polyurethane. In an embodiment, the core does not contain polyester.

The core may be present in an amount of from about 20 wt. % to about 80 wt. %, based on the total weight of the multi-functional damage responsive polymeric fiber.

The core of the multifunctional damage responsive polymeric fiber provides mechanical properties, such as load-bearing and energy absorption to the BioFiber. In addition, it provides a proper environment for proliferation of endospores infused within the hydrogel sheath. For example, when polyester (PES) and polyvinyl alcohol (PVA) fibers are selected as core material, they have demonstrated robust mechanical properties and bio-compatibility characteristics.

The polymeric fiber used as the core may have a fiber length to a fiber diameter ratio of from about 20 to 80, or from about 20 to about 60, or about 40.

The polymeric fiber used as the core may have a fiber length of from about 10 mm to about 100 mm, or from about 10 mm to about 70 mm, or from about 10 mm to about 60 mm.

The polymeric fiber used as the core may have a fiber diameter of from about 300 μm to about 2500 μm, or from about 400 μm to about 2000 μm or from about 500 μm to about 1750 μm.

### Endospore-Laden Hydrogel Layer

The multi-functional damage responsive polymeric fiber of the present invention may include a hydrogel layer. The core is coated by a hydrogel layer which may include, for example, an anionic polymer, a crosslinking agent, and one or more endospores as the bacteria healing agent.

The term “anionic polymer” refers to any polymer, preferably of biological origin, with a net negative charge, including in said definition those anionic polymers on which modifications have been made such as enzymatic or chemical fragmentation or derivatization. Suitable examples of the anionic polymer may include polysaccharides, such as hyaluronic acid, colominic acid, polysalic acid, chondroitin, queratane, dextrans, heparin, carrageenan, furcelerans, alginates, agar agar, glucomannan, gums, pectins, cellulose, starches, and sorbitan esters. For example, the hydrogel layer may comprise an anionic polymer including sodium alginate.

When the hydrogel layer comprises sodium alginate as the anionic polymer, the sodium alginate may be present in an amount that provides a sodium concentration of from about 2% w/v to 8% w/v, based on a total volume of the hydrogel layer.

The hydrogel layer may be formed by coating the polymeric core material using a reel-to-reel system to form a coated polymeric material. The method of coating the polymeric core material may use a speed of 10 rot/sec to 100 rot/sec, or from 20 rot/sec to 100 rot/sec, or from about 50 rot/sec to 100 rot/sec.

The hydrogel layer may be crosslinked with a cationic crosslinking agent. The cationic crosslinking agent may be divalent, for example, the cationic crosslinking agent may be selected from calcium chloride, calcium acetate, and combinations thereof. The method for crosslinking the hydrogel layer may comprise of passing the coated polymeric material through a bath solution of one of the suitable crosslinking agents to form a crosslinked coated polymeric material. Following the crosslinking step, the crosslinked coated polymeric material may be dried using a variety of well-known methods in the art.

The hydrogel layer may include therein an endospore. The germination of the endospore is triggered by the presence of carbon and a nutrient source, for example, yeast extract and urea. Urea hydrolysis-driven MICCP may be initiated by the presence of the crosslinking agent, for example, calcium chloride, calcium acetate, or combinations thereof. Suitable examples of endospores that may be incorporated into the hydrogel layer may include Lysinibacillus sphaericus strain MB284. The endospore can be a synthetic or naturally occurring endospore. The endospore may be present in a typical concentration of 109 cells/mL.

The hydrogel layer may have a thickness of from about 0.5% to about 20% of the thickness of core fiber before swelling. The thickness of the hydrogel layer may be adjusted by changing the concentration of the anionic polymer. For example, increasing the concentration of sodium-alginate resulted in higher loads of hydrogel onto the core-fiber, which corresponded to higher swelling capacity and higher endospore concentrations in the BioFibers. Through a parametric study, it was determined that deviations from neutral pH may cause a decrease in the hydrogel swelling ratio, up to 57% for acidic and 72% for basic conditions.

### Polymer Shell

The hydrogel layer is encapsulated by a polymer shell. Suitable none limiting materials for forming the polymer shell may include nitrocellulose (NITR), epoxy Resin (ER), polymethylmethacrylate (PMMA), polyvinylidene fluoride (PVDF), cyanoacrylate adhesive (CYA), polystyrene (PS), polylactic acid (PLA), and combinations thereof.

The multi-functional damage responsive polymeric material once coated with the polymer shell may have a diameter of from about 300 μm to about 3000 μm, or from about 400 μm to about 2500 μm.

The shell may have a shell thickness of approximately 10% to 30%, or from about 10% to about 25% of the core fiber thickness.

For example, the polymer shell may include polystyrene and polylactic solution, in a ratio of 50:50 wt. %.

The polymer shell may be formed by a variety of polymerization methods, which may include free radical polymerization, or dipping the dried crosslinked coated polymeric material into a polymeric mixture to form the multi-functional damage responsive polymeric fiber.

The polymer shell may be formed by layers, wherein the polymer shell may include one layer, one or more layers, or two or more layers, or three or more layers, or four or more layers, or five or more layers, wherein each of the layers may be the same material or different.

The uncured polymeric mixture may be prepared in the presence of a solvent or initiator. Suitable none limiting examples of solvents and initiators may include, aliphatic polyamine hardener, benzoin, benzoyl peroxide, dimethoxypropane, and chloroform.

The polymer shell preferably has a surface that possesses no or little porosity. It is preferred that the polymer shell does not include a porous structure since this can adversely impact the shell survivability against the fluid ingress. If there is any porosity in the structure, porous areas should not be interconnected to ensure impermeability.

The uncured polymeric mixture may be cured at room temperature for 10 minutes to 24 hours, or from about 20 minutes to 24 hours, or from about 30 minutes to 2 hours, or for about 30 minutes, or for about 1 hour, or for about 2 hours, or for about 24 hours. Alternatively, the uncured polymeric mixture may be cured for about 30 minutes to 3 hours, or from about 1 hour to about 2 hours using ultraviolet radiation, with a wavelength of 340 nm, and an irradiance of 0.89

$$\frac{W}{{m}^{2} \cdot nm}$$

As an example, Table 1 shows the possible combinations of shell material, possible solvents/initiator, and curing/polymerization conditions.

TABLE 1

| | | | Curing/Polymerization |
|---|---|---|---|
| | Shell Material | Solvent/Initiator | Condition |
| 1 | Nitrocellulose (NITR) | — | 1 hour, RT* |
| 2 | Epoxy Resin (ER) | Aliphatic | 24 hours, RT |
| | | Polyamine | |
| | | Hardener | |
| 3 | Polymethylmethacrylate | Benzoin | 2 hours |
| | (PMMA) | Benzoyl peroxide | Ultraviolet radiation |
| | | | (wavelength: 340 nm, |
| | | | irradiance: 0.89 |
| | | | W/(m² · nm) |
| 4 | Polyvinylidene Fluoride | Dimethoxy | 1 hour, RT |
| | (PVDF) | propane (DMP) | |
| 5 | Cyanoacrylate Adhesive | — | ½ hour, RT |
| | (CYA) | | |
| 6 | Polystyrene (PS) | Chloroform | ½ hour, RT |
| 7 | Polylactic Acid (PLA) | Chloroform | ½ hour, RT |

*RT: Room Temperature (23 ± 1° C.)

For example, the incorporation of the polystyrene/polylactic acid copolymer shell coating is suitable for introducing the necessary damage-responsive characteristic to the BioFibers. The copolymer coating functions as a protective barrier, preventing the release of endospores prior to the occurrence of cracks. The thickness of the hydrogel and shell coating may be tailored to achieve the minimum value that meets the performance requirements, for example, hydrogel high swelling ratio and shell survivability against fluid ingress.

For example, the polymer blend (1:1 wt. %) of polylactic acid and polystyrene dissolved in chloroform demonstrated a suitable shell coating for BioFibers as it provided a uniform shell with low porosity. Different copolymer/solvent ratios resulted in different shell coating ratio, affecting the shell survivability against fluid ingress and mechanical forces applied during mixing with the matrix. Shell thicknesses of 35% and 18% of the core fiber thickness were required to pass 1 hour fluid ingress survivability test on the PES and PVA core-fiber, respectively. For simulated concrete casting, a shell including PVA with 18% w/v, 1-layer provided more abrasion resistance, and a 90% and 80% survivability under hand mixing and mechanical mixing, respectively.

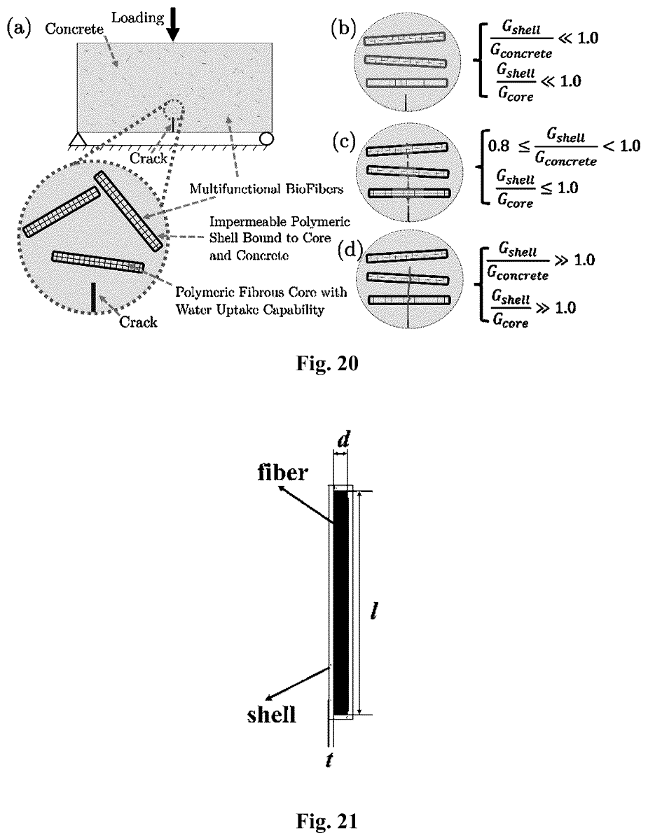

In another aspect, the present invention relates to computation damage models to predict the fracture response of BioFibers in a quasi-brittle material as a matrix. A quasi-brittle material was used as an example to provide predictive simulations to indicate that BioFibers are capable of enhancing fracture behaviour of quasi-brittle materials. In the finite element models, fibers have been considered with a coating, also referred to as the polymer shell herein. (FIG. 21). These damage models are to guide the manufacturing process of BioFibers to tune the material properties and mechanical response of BioFibers in order to achieve desired fracture properties, for example, matrix fracture should result in strain fracture of the shell and release of the bacteria healing agent (one or more endospores) while the core should remain strong to bridge the crack. Additionally, the subsequent unbroken fibers at the tip of the crack should help to restrain and limit the crack growth in the quasi-brittle material. As such, it is theorized that a less ductile fiber-shell material would provoke the strain rupture of shell. FIG. 20 illustrates the ideal fracture energy release rate of the shell vs. core vs. matrix to achieve the right balance when incorporated in a quasi-brittle material.

For example, the various components ideally meet the following criteria:

$$0.8 \leq \frac{{G}_{shell}}{{G}_{matrix}} < 1. and \frac{{G}_{shell}}{{G}_{core}} \leq 1. \qquad Equation A$$

wherein Gₛₕₑₗₗ is defined as the fracture energy release rate of the polymer shell, Gₘₐₜᵣᵢₓ is defined as the fracture energy release rate of the matrix, and G_{core} is defined as the fracture energy release rate of the core.

This numerical analysis was conducted utilizing phase-field fracture framework. As the choice of the right shell material property is of particular importance, different material mismatch cases for the critical energy release rate of the shell, compared to that of the core fiber material and hydrogel laden endospore matrix, have also been considered. In addition to the material properties, the geometrical features of BioFibers, i.e., the shell thickness and the ratio of fiber length to diameter and their effects on the fracture resistance of the BioFibers have also been analyzed. Moreover, the application of two different fibers, for example, polyester fiber and polypropylene fiber provides an almost 10 times higher critical energy release rate in comparison to non-reinforced simulations. All the structures have been analyzed under three loading conditions: tensile loading, compressive loading, and three-point bending. In order to judge what material property mismatch and configuration perform best, the values of peak force and absorbed energy of each structure in each case study have been taken into consideration and compared with those of other structures.

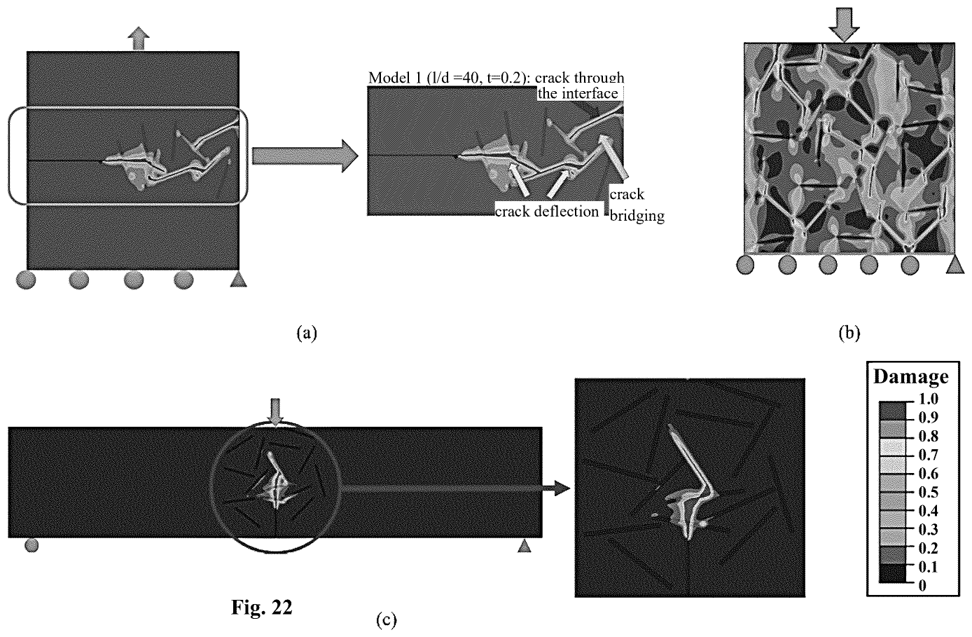

It was demonstrated that the most favorable performance and configuration depended on the geometry and also the material mismatch property (FIG. 22). Under tension, BioFibers with the lowest fiber length to diameter ratio exhibit the highest peak force and absorbed energy in the case of polyester fiber. The same observation was made for all models and material sets under compressive loading. Under three-point bending loading condition, for the cases of polyester fiber, similar results were obtained as the lowest fiber length to diameter ratio showed the best mechanical response. Accordingly, the results confirmed that shell material had a dominant effect on the fracture response of the structure under this loading condition. More precisely, a shell whose energy release rate is slightly below that of the matrix would promote cracks to propagate through the interface, causing the debonding of the fibers and a considerable rise in absorbed energy. Thus, not only was the absorbed energy higher, but more fibers remained intact, and there were multiple smaller cracks through the matrix and shell mostly, which is easier to heal. Polypropylene fibers also managed to increase the peak forces which the BioFibers underwent under different loading conditions. Moreover, by the use of this fiber type, many of the fibers remained intact and managed to deflect the cracks through the fiber-shell interface.

### EXAMPLES

The following examples were carried out using a variety of materials to tailor the processing-compositions-structure properties of the developed BioFibers to achieve multifunctionalities.

### 2.1.1 Polymeric Core-Fiber

The PVA fibers used throughout the examples were commercial water-insoluble fibers (NYCON RF4000) produced mainly for concrete applications. The PES fibers were purchased from Unifi, as recycled polyester. The material/geometrical properties of the core-fibers used in the Examples are listed in Table, as reported by the manufacturer.

One of the primary functions of the core-fiber is to integrate load-bearing and energy absorption to the BioFiber, however, it is important to ensure that it provides a proper environment for proliferation of endospores which are infused within the hydrogel sheath. In the following examples, polyester (PES) and polyvinyl alcohol (PVA) fibers were selected as the core-fibers, which provided robust mechanical properties and bio-compatibility characteristics. The PVA fibers used in this study were commercial water-insoluble fibers (NYCON RF4000) produced mainly for concrete applications. PES fibers were purchased from Unifi, as recycled polyester. The material/geometrical properties of the core-fibers used in these examples are listed in Table 2, as reported by manufacturer.

TABLE 2
Polymeric core-fiber material/geometrical properties

| | | Polyvinyl |
|---|---|---|
| Properties | Polyester (PES) | alcohol (PVA) |
| Length (mm) | Varied | 30 |
| Diameter, Average (μm) | 301 | 611 |
| Diameter, Standard Deviation (μm) | 20 | 19 |
| Density (g/cm³) | 0.63 | 1.44 |
| Tensile Strength (MPa) | 181 | 800 |
| Elastic Modulus (GPa) | 0.8 | 23 |
| Morphology | Multi-Filament | Mono-Filament |

### 2.1.2 Endospore-Laden Hydrogel

The hydrogels were loaded with Lysinibacillus sphaericus strain MB284 as the endospore bio-healing agents. Germination of the endospore was triggered by the presence of carbon and nutrient source, i.e., yeast extract and urea, and urea hydrolysis-driven MICCP was initiated by the presence of a calcium source. Yeast extract was purchased from Sigma-Aldrich (CAS:8013-01-2), and the urea was purchased from Alfa Aesar (CAS:57-13-6).

Previous studies revelated that thermal shock endosporulation methods resulted in production of endospores that were capable of surviving, germinating, and growing under inhospitable conditions [28]. In the thermal shock method, Lysinibacillus sphaericus vegetative cells were first incubated into the culture medium, which included yeast extract (20 g/L) and urea (20 g/L), for 24 hours to reach the exponential growth phase of the cells. Next, the vegetative cells were washed three times with 1 molar of Phosphate Buffer Solution (PBS) and inoculated into a Minimal Salt Media (MSM) [28]. Following this, the cells were incubated for 30 minutes in a boiling water bath followed by 30 minutes in an ice water bath.

Other studies have shown that the endospores produced through the thermal shock method can germinate in harsh alkaline conditions (for example, a pH of 12), high salinity environments (up to 100 g/L), and under freeze-thaw cycles (a temperature of −10 to 60° C.) [28]. For the hydrogel, sodium-alginate (Na-Alg) was selected as the anionic polymer, or prepolymer, and aqueous calcium chloride and calcium acetate was selected as the source of divalent cations. The sodium-alginate was purchased from Sigma-Aldrich (CAS: 9005-38-3), calcium chloride dihydrate from MP Biomedical (CAS: 10035-04-8), and calcium acetate monohydrate from CHEM-IMPEX INT'L INC (CAS: 5743-26-0).

### 2.1.3 Polymeric Outer Shell Layer

To integrate damage-induced healing mechanism into the BioFiber, a strain-responsive polymeric-based shell coating was included as the outer layer on the hydrogel coated core-fiber. The following polymeric materials were considered for the BioFiber shell coating along with the following conditions.

| | | | Curing/Polymerization |
|---|---|---|---|
| | Shell Material | Solvent/Initiator | Condition |
| 1 | Nitrocellulose (NITR) | — | 1 hour, RT* |
| 2 | Epoxy Resin (ER) | Aliphatic | 24 hours, RT |
| | | Polyamine | |
| | | Hardener | |
| 3 | Polymethylmethacrylate | Benzoin | 2 hours |
| | (PMMA) | Benzoyl peroxide | Ultraviolet radiation |
| | | | (wavelength: 340 nm, |
| | | | irradiance: 0.89 |
| | | | W/(m² · nm) |
| 4 | Polyvinylidene Fluoride | Dimethoxy | 1 hour, RT |
| | (PVDF) | propane (DMP) | |
| 5 | Cyanoacrylate Adhesive | — | ½ hour, RT |
| | (CYA) | | |
| 6 | Polystyrene (PS) | Chloroform | ½ hour, RT |
| 7 | Polylactic Acid (PLA) | Chloroform | ½ hour, RT |

*RT: Room Temperature (23 ± 1° C.)

### 2.2 BioFiber Manufacturing

A surface functionalization method defined as instant immersion, was used to manufacture the BioFibers. This technique included soaking of the core-fibers, e.g. PES and PVA, in three solution baths, for example 1) Na-Alg/endospore solution, 2) calcium crosslinker solution, 3) polymeric-based coating solution or uncured/pre-crosslinked liquid. For the first step, loading an endospore-laden hydrogel on the core-fiber, Na-Alg powder was mixed with an endospore suspension with a concentration of 10⁵ cells/ml, and gently stirred for 1 hour. Calcium chloride and calcium acetate solutions were prepared in deionized water. The 30 mm length core-fibers were first soaked in the Na-Alg/endospore solution and followed by soaking in the calcium crosslinker solution to trigger the ionic crosslinking process. In both stages, the immersion in the solution was immediate, with no delay in the soaking process. After a sheath of endospore-laden hydrogel was created on the core-fibers, the fibers were set to dry under ambient condition (23° C.) for 24 hours. In order to tune the coating thickness and swelling capacity of the hydrogel, the concentration of the alginate solution was changed as 2, 4, and 8 weight-to-volume (w/v), based on the total volume of the hydrogel layer. In the examples, the concentration of calcium crosslinker solution kept constant at 0.259 M.

The next step included applying an impermeable strain-responsive polymeric shell coating on the hydrogel coated fibers. Different polymeric solutions were prepared based on the type of shell material. In the case of lacquers polymers (e.g., Nitrocellulose), the shell coating process was carried out by evaporating solvents used in the formulation of lacquers. The shell bath contained the polymer/solvent solution.

For reactive prepolymers and polymers, e.g., epoxy resin, the shell bath contained the prepolymers mixed with co-reactants, of which the polymerization occurred through an exothermic reaction between reactive and co-reactants materials. For methyl methacrylate (MMA) polymerization, free radical polymerization (FRP) technique was used to create a shell layer on the BioFibers. For this method, benzoyl peroxide and benzoin (each 0.5 g in 10 mL of MMA) were used as the photo-initiator, decomposed into free radicals under irradiation of ultraviolet (UV) light, to synthesize a polymethylmethacrylate (PMMA) film on the hydrogel coated fiber. Benzoyl peroxide under UV irradiation resulted in the formation of two benzoyloxy radicals and benzoin generated benzoyl and α-hydroxybenzyl radicals as an initiation stage for the FRP mechanism.

In the next stage, propagation, the free radicals attached to the MMA monomer, building polymer chains. The polymer chain length and the termination stage of FRP depended on the photo-initiator concentrations. Since different polymerization methods with various polymeric materials were used, different drying, curing, and polymerization conditions were applied, as shown in Table 2.

### 2.3 Experimental Program

To evaluate the performance of the manufactured BioFiber, a series of tests were carried out to test the functionality of the hydrogel, shell coating, and the overall BioFiber, as shown in Table 1.

TABLE 1
Experimental program for BioFiber performance assessment

| Experiment | Objective |
|---|---|
| Optical Microscopy | To visually investigate the hydrogel and outer shell |
| | coating morphology on the core-fibers |
| Scanning Electron | To perform microstructural analysis on the BioFiber |
| Microscopy | after each surface coating applied on the core-fiber |
| Gravimetric | To determine the swelling capacity of endospore- |
| Swelling Analysis | laden hydrogel in different conditions |
| Fluid Ingress | To investigate the impermeability of outer shell |
| Survivability Test | coating against ingress of aqueous solution |
| Abrasion Resistance | To study the abrasion resistance of the shell coating |
| | under simulated casting process |
| Thermogravimetric | To quantify the amount of precipitated calcium |
| Analysis | carbonate via MICCP activity |

### 2.3.1 Coating Morphology Assessment

The thickness and uniformity of the hydrogel laden endospore coating are factors that can influence the overall functionality and cost of the BioFiber. In these examples, optical microscopy was used to determine the thickness and uniformity of the endospore-laden hydrogel coating loaded on the polymeric core-fiber. The hydrogel thickness to core-fiber diameter ratio was calculated using equation 1:

$${t}_{Hgel \%} = ( {t}_{Hgel} / {D}_{cf} ) \times 100 \qquad Eq . 1$$

where t_{Hgel %} is the hydrogel thickness ratio in percentage, t_{Hgel} is the hydrogel thickness, and D_{ef} is the core-fiber diameter. In addition, the weight of hydrogel loaded on the core-fibers was calculated using equation 2:

$${W}_{Hgel} ( mg / mg / cm ) = ( {W}_{F H} - {W}_{F} / {W}_{F} ) / {L}_{F} \qquad Eq . 2$$

where W_{Hgel} is the normalized weight of hydrogel loaded on the core-fiber to the length of the fiber (mg/mg/cm), W_{F} is the initial weight of core-fibers, W_{FH} is the weight of the hydrogel coated core-fibers, and L_{F} is the length of the core-fiber. To ensure repeatability of measurements, 30 samples were used in this test, with 10 measurements over the length of each fiber. For the hydrogel coating morphology study, the hydrogel was prepared using 2, 4, 8 w/v of Na-Alg crosslinked with calcium acetate (CA) and calcium chloride (CC) maintained at a constant concentration of 0.259 M.

Similar to hydrogel coating, the morphology assessment of the shell coating finish on the BioFibers is important to ensure manufacturing of a high-performance BioFibers. The shell coating morphology on the BioFibers controls the interfacial properties between the concrete and the fibers, crack-bridging functionality, and the protection of inner layers. To perform the morphology assessment, an optical microscopy technique was utilized to determine the shell coating uniformity and the thickness ratio. In this experiment, the hydrogel was prepared using 8 w/v of Na-Alg, crosslinked using calcium acetate with molarity of 0.259 M. For measuring the coating thickness for each shell material, five replicate samples were prepared with 10 measurements reading on each sample. The shell coating thickness to hydrogel coated core-fiber was calculated based on the thickness measurement using Equation 3:

$${t}_{Shell \%} = ( {t}_{Shell} / {D}_{hcf} ) \times 100 \qquad Eq . 3$$

where t_{Shell %} is the shell coating thickness ratio in percentage, t_{Shell} is the shell coating thickness, and D_{hcf} is the diameter of the core-fiber with hydrogel coating core-fiber.
   2.3.2 Microstructural analysis

The BioFibers were tested for microstructural analysis after the manufacturing process, and before/after MICCP activation. The samples were coated with a 12 nm thick layer of 80/20 platinum/palladium using a Cressington 208 sputter coater (Ted Pella, Inc., Redding, CA), since the materials used for BioFiber manufacturing are nonconductive. Scanning electron microscopy (SEM) was then performed using a Zeiss Supra 50/VP to observe fiber morphology on all samples.

### 2.3.3 Endospore-Laden Hydrogel Swelling Performance

Swelling capacity is a desired feature of hydrogels since it impacts the MICCP urea hydrolysis-driven chemical pathways by controlling the amount of water delivered to the endospores. The swelling ratio is defined as the quantity of mass gain of the hydrogels when exposed to aqueous solutions at various time intervals. To determine the swelling ratio, gravimetric analysis was performed on the endospore-laden hydrogel coated core-fibers at a dry and wet state. First, dry pre-weighted endospore-laden hydrogel coated core-fiber samples were immersed in excess of swelling medium. After certain exposure time intervals, the samples were removed from the swelling medium, with excess solution being cleared away from each sample surface by wiping with a soft tissue cloth, and then weighed immediately. The swelling ratio based on these weights were calculated according to the following equation:

$${S}_{r} = ( {W}_{s} - {W}_{d} ) / {W}_{d} \qquad Eq . 4$$

where Sᵣ is the swelling ratio, Wₛ and W_{d} are the weight of the samples in swollen and dry state, respectively. Since the swelling capacity is highly affected by the hydrogel structures, a parametric study was performed on the effects of the Na-Alg concentration, i.e., 2, 4, 8 w/v, and the cation crosslinker type, CA and CC, on the swelling ratio. In addition, the pH of the aqueous solutions was modified to 3, 7, 13 to study the swelling ratio sensitivity to acidic, neutral, and basic conditions. The swelling ratio was calculated at consecutive time intervals of 2 minutes, 5 minutes, 10 minutes, and 15 minutes. For each set of tests, five replicates were used to ensure the measurement repeatability.

### 2.3.4 Fluid Ingress Survivability Test

Impermeability test was performed to investigate whether the shell coating can protect the endospore-laden hydrogel from unwanted access to moisture and/or aqueous solution. The impermeability test was designed based on monitoring the pH-sensitive indicator color change as BioFibers with different shell coating materials were exposed to an aqueous medium. Phenolphthalein, an organic-based acid-base indicator, was blended with the Na-Alg/endospore solutions at a concentration of 0.1 w/v, and hydrogel and shell coating on the core-fibers was performed with similar steps, as discussed earlier. Phenolphthalein is colorless at pH below 8.5, reaching pink to deep purple color in a medium with a pH above 9. Using this feature, the BioFibers were exposed to high alkaline synthesized pore solution for time intervals of 1 and 2 hour(s). In the case of permeable shell coating, the solution reached the inner layers, exposing phenolphthalein to high pH and color change. Observing pink/purple color on the surface of the BioFibers was considered as an indication of permeable shell coating. In the case of impermeable shell coating, no color change was observed on the surface or in the exposed solutions. The color change observation was performed using an optical microscope.

### 2.3.5 Abrasion Resistance Performance

The primary purpose of developing BioFibers is to incorporate them into a quasi-brittle matrix to enable self-healing, thus it is preferred to test the survivability of shell coating during blending manufacturing. To test the BioFibers ability to handle stress, the BioFibers were subject to a variety of shear mixers which apply shear loading stresses to the BioFibers.

In the manufacture of the BioFibers, similar to fluid ingress survivability test, 0.1 w/v of phenolphthalein was blended with hydrogel. The shell materials used for this experiment were those materials shortlisted based on the impermeability test results. Using a typical fiber reinforced quasi-brittle mortar sample mixture (as shown in Table 2), the BioFibers were incorporated into the mixture without binder materials. The binder materials were excluded from the test to ensure the easy removal of the BioFibers after the casting stage to allow for further study the abrasion resistance, and to investigate any color change in the added phenolphthalein. The amount of binder that was excluded from the concrete mix was replaced with standard sand, to introduce a more severe casting condition. For each set of tests, 20 units of BioFibers were employed, mixed with standard sand and water using two methods: manual hand mixing and mechanical mixing using a shear mixer. After the mixing stage, the samples were separated from the other components to observe whether they remained intact or fractured. The survivability (%) was defined based on the number of intact BioFibers over the total number of BioFibers used. The BioFiber mixing process with mortar components were performed with two methods: (a) 2-minute hand mixing (HM), and (b) 2-minute mechanical mixing (MM) using a vacuum shear mixer with 350 rps. The hand mixing and mechanical mixing were used to study the impact of using different mixing methods on the casting survivability of the BioFibers.

After the mixing stage, the samples were poured on a tray, and the BioFibers were separated from water and sand. Then, the BioFibers were exposed to alkaline synthesized pore solution with a pH of 13 for 1 and 2 hours. Finally, the BioFibers were examined under an optical microscope to detect color change on the surface of the BioFibers. Since the impermeable shell materials were used for this experiment, any color change or release was associated with the shell fracture during the casting process.

TABLE 2
Mortar mix design for simulated casting process

Volume (%)

| Material | Typical | Adjusted |
|---|---|---|
| Quasi-Brittle Material | 25.31 | 0.00 |
| Water | 33.69 | 33.69 |
| Standard Sand | 40.00 | 65.31 |
| Fiber | 1.00 | 1.00 |
| Total | 100.00 | 100.00 |

### 2.3.6 Microbial Induced Calcium Carbonate Precipitation (MICCP) Performance

Previous experiments were designed to evaluate the performance of BioFiber elements, i.e., hydrogel and shell. In this stage, the objective was to conduct a quantitative/qualitative analysis of calcium carbonate precipitation as an end-product of urea hydrolysis-driven chemical reactions to heal quasi-brittle materials. In order to promote self-healing process, the fractured and intact, as the control sample, BioFibers were exposed to a solution media containing yeast extract, urea, and calcium acetate (each with a concentration of 20 g/L) for 30 hours. No MICCP activity was hypothetically expected in the case of intact BioFibers. In the fractured BioFibers, the hydrogel absorbed water and released endospores to the media.

Next, the endospores interacted with the organic carbon source, which act as nutrients to the endospores, allowing them to initiate germination and outgrowth stages. Calcium acetate provided a source of calcium ions for the final precipitation of calcium carbonate. To quantify the amount of precipitated calcium carbonate, thermogravimetric analysis (TGA) was performed on the solid residue after MICCP termination. After exposing the BioFibers to a calcium/carbon source, the solution media was centrifuged for 9 minutes at 7830 rpm and a temperature of 25° C. to terminate the MICCP process and to obtain the residue. The residue was maintained at 105° C. for 1 hour to remove the remaining water/moisture from the samples. Then, the solid residue was ground to collect the particles with a diameter of less than 75 μm. 20-30 mg of the samples were deposited at a high-temperature platinum pan, and TGA tests were conducted at 30-900° C. with a ramp rate of 10° C./min.

### 3. Results and Discussions

### 3.1 Hydrogel Physical/Morphological Properties

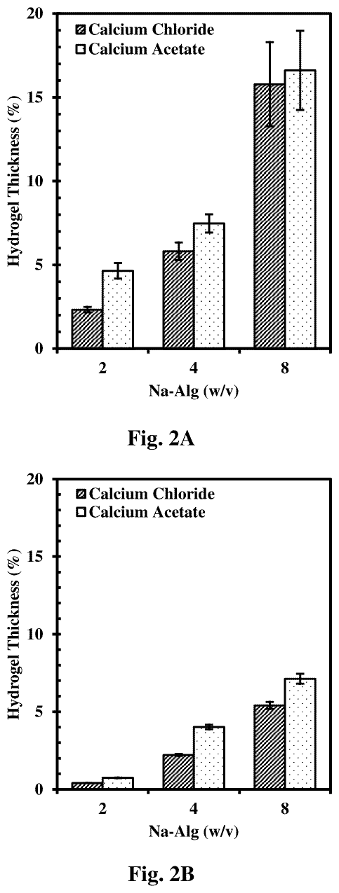

The hydrogel layer thickness was adjusted by changing the concentration of anionic polymers, or prepolymers, for example, Na-Alg. The thickness measurements were performed using an optical microscope, and the results are shown in FIG. 2A. For both PES and PVA core-fiber, an increasing trend on the hydrogel thickness was observed as the concentration of Na-Alg increased. This trend was associated with the increase in prepolymer viscosity as the prepolymer concentration increased. Higher prepolymer concentration increased hindrance and friction among the polymer chains, increased the prepolymer solution viscosity, and resulted in higher adherence of Na-Alg into the core-fibers. For instance, the hydrogel crosslinked with calcium acetate on the PES led to hydrogel thickness of 4.65% and 16.61% for Na-Alg concentrations of 2 and 8 w/v, respectively. From a gelation time perspective, the increase in prepolymer concentration leads to slower rearrangement of the polymer chain conformations. However, since the crosslinking reaction was completed in a single instant soaking step in a crosslinker bath, the gelation time was not a key factor affecting BioFiber manufacturing. Although the concentration of cation crosslinker solutions was constant at 0.259M of Ca²⁺, the hydrogel coating formation on the core-fiber was slightly thicker in all cases as calcium acetate solution was used compared with calcium chloride. The hydrogel formation is dependent on the calcium ion diffusion rate into the alginate polymer chain, calcium distribution homogeneity/inhomogeneity and clogging issues, resulting in a slight difference between the hydrogel prepared using calcium acetate and calcium chloride.

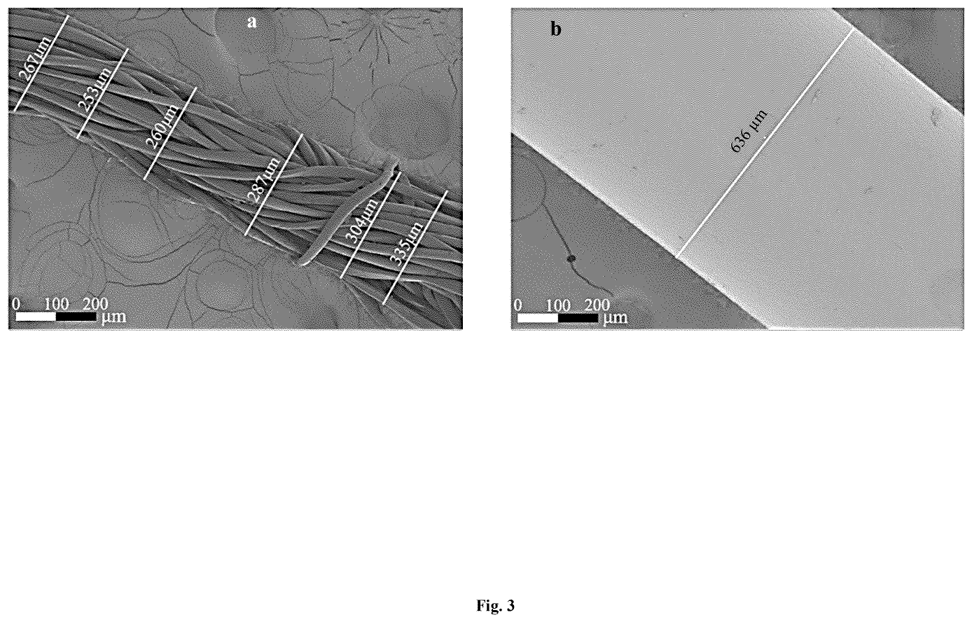

In terms of the type of core-fiber effects on the hydrogel coating thickness, the results revealed considerable differences between PES and PVA, as higher hydrogel thickness was observed in PES. The differences can be due to fibers morphology, physical properties and chemical compositions. Regarding the physical differences between PES and PVA, the cross-section structures played a key role in the amount of hydrogel loaded on the core-fibers. PES is a multi-filament crimped yarn, wherein the PVA fiber is a monofilament (Table). In addition, PES fibers have asymmetric crimped structures with nonuniform cross-section area over the length of the fibers, while PVA fibers have round and symmetrical cross-section structures, as observed in SME images (FIG. 3). PES fibers crimped structure provided a higher surface area for the prepolymer solution to adhere, leading to higher hydrogel coating thickness.

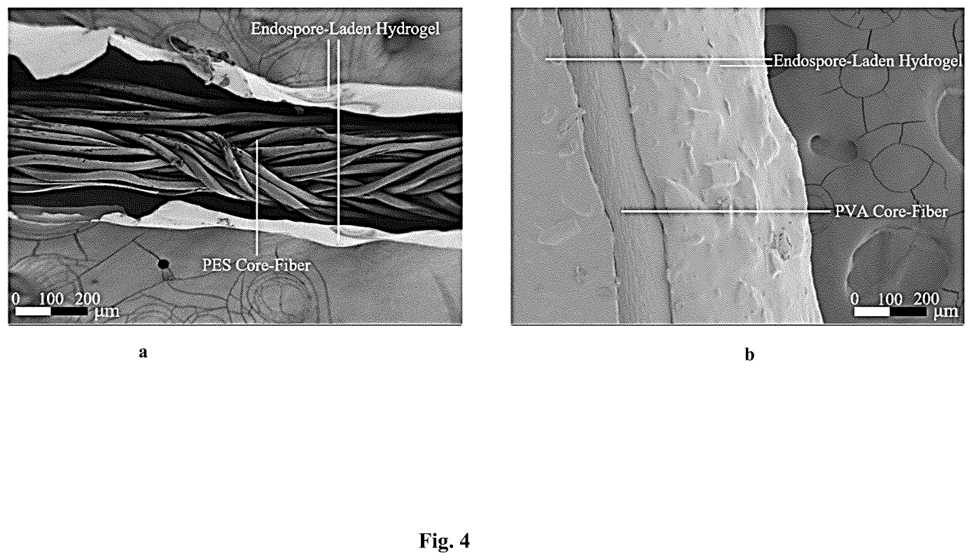

In addition, the hydrogel penetrated the PES filament inter-spaces, supplying more hydrogel attachment. On the other hand, the PVA fibers have smooth surfaces and therefore provided less surface area and no opportunity for the hydrogel to interpenetrate into the monofilament. However, the hydrogel thickness results indicated less variance in the PVA fiber than PES, which can be associated with the uniformity of PVA diameter along its length, resulting in a more uniform hydrogel coating. In FIG. 4, hydrogel coatings of PES and PVA fibers were cut to produce SEM images showing the interface between hydrogel and core-fibers. It was concluded that after completion of hydrogel crosslinking and drying, the hydrogel had higher interfacial interaction with PVA rather than PES. In terms of chemistry, both PES fibers are made of hydrophobic compounds, which decrease the compatibility with hydrophilic alginate. On the other hand, PVA fibers contain hydrophilic (hydroxyl group) and hydrophobic groups (acetate group), based on the degree of hydrolyzation during manufacturing. Based on SEM images shown in FIG. 4, PVA fibers indicated higher chemical compatibility with calcium-alginate hydrogel due to hydrophilicity of the hydroxyl groups, compared to lesser chemical compatibility observed in hydrophobic PES fibers.

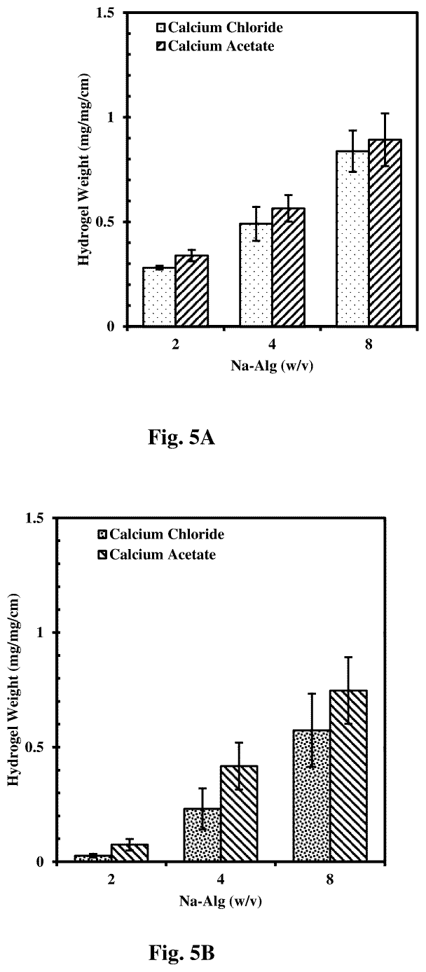

In addition to hydrogel thickness, the weight of the loaded hydrogel on the core-fibers was calculated, as shown in FIG. 5. The significance of presenting the hydrogel weight normalized per length of the core-fibers was for the applications where the weight of hydrogel would be of higher priority than the thickness. In terms of self-healing, the mass of hydrogel controls the swelling ratio and the amount of loaded bio-self-healing agents on each unit length of the BioFibers. The trend of hydrogel weight change was similar to the thickness considering the Na-Alg concentration, crosslinker type, and the core-fiber materials.

### 3.2 Hydrogel Swelling Performance

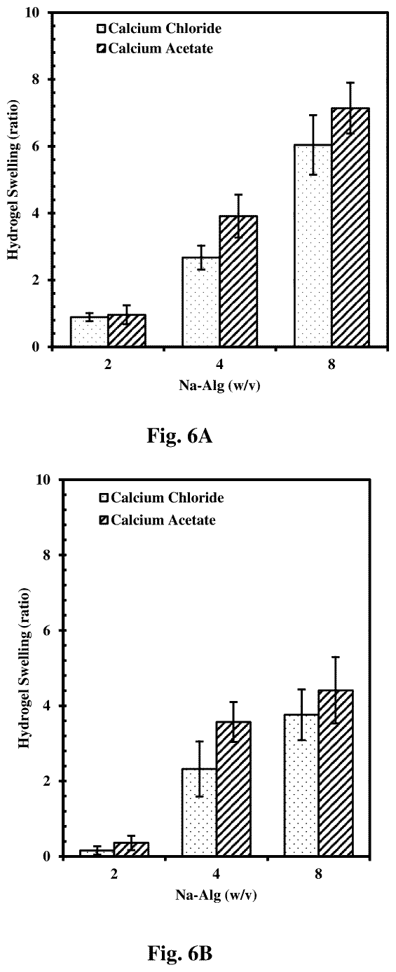

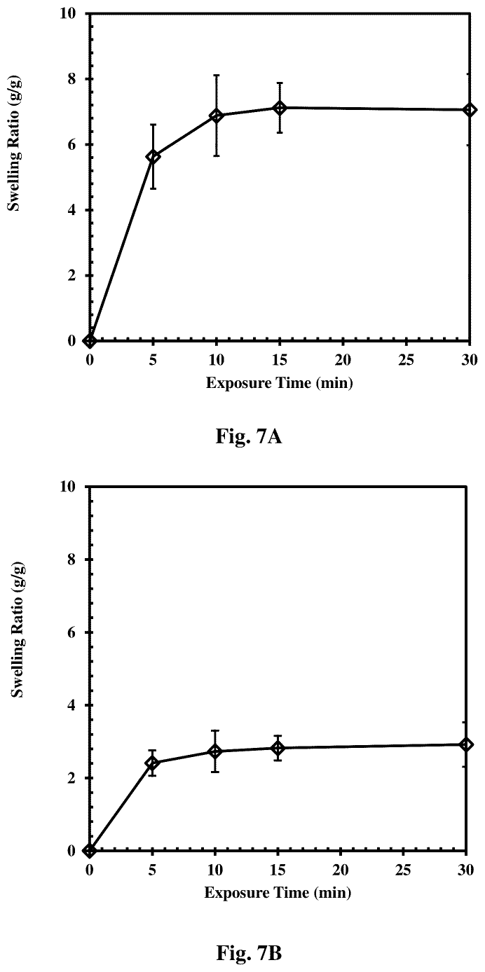

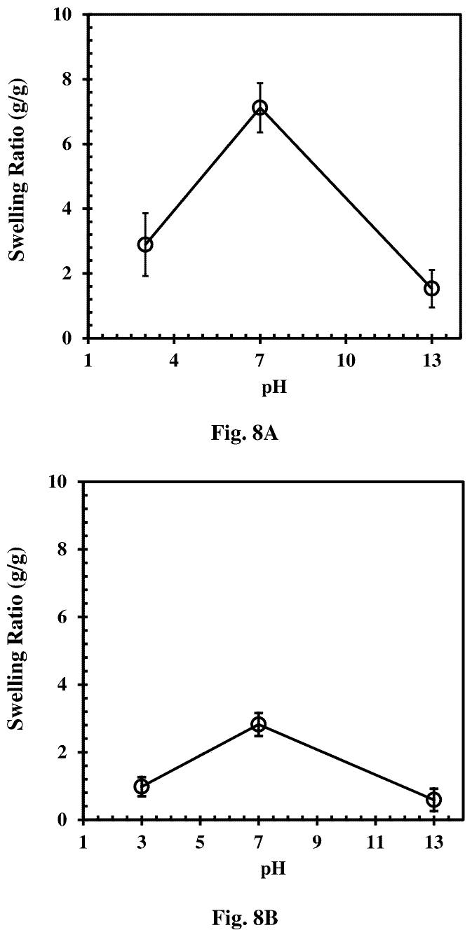

The swelling capacity of the hydrogel-coated core-fibers was measured through gravimetric analysis and presented in FIG. 6 to FIG. 8. The effects of Na-Alg concentration, cation crosslinker type, time of exposure to the solution, and pH of the solution were investigated. The results revealed that the hydrogel crosslinked with higher Na-Alg concentration and CA showed a higher swelling ratio (FIG. 6) since a higher weight of hydrogel was loaded on the core-fibers in these conditions. For instance, the core-fibers coated with 8 w/v Na-Alg crosslinked with CA showed a swelling ratio (g/g) of 7.14 for PES and 4.41 for PVA, as the samples were exposed to DI water for 15 minutes. These results revealed that the swelling ratio of crosslinked alginate can be mainly tailored by using different prepolymer concentrations. In FIG. 7, the effects of time of exposure on the swelling ratio of the hydrogel was studied under time interval of 5, 10, 15, and 30 minutes. The samples with crosslinked hydrogel using 8 w/v Na-Alg and CA were employed for this parametric study. The results also indicated that the swelling ratios increased as the exposure time increased to 10 minutes. The swelling ratios reached a plateau after 10 minutes of exposure, indicating that the polymer chain arrived at the maximum liquid absorption capacity. Since the crosslinked alginate swelling capacity is pH sensitive, a parametric study was conducted using acidic, neutral, and basic aqueous solutions with pH of 3, 7, and 13. Ethanoic acid (CH₃COOH) was used as a solution with acidic pH, and sodium/potassium hydroxide solution was used for synthesizing basic pH. FIG. 8 Figuredisplays the variation of the hydrogel swelling ratio as the pH of the exposed solution shifted towards acidic or basic solutions. Under acidic conditions, the limited expansion ratio can be attributed to the strong repulsion between anion-anion COO− groups. However, in basic conditions (specifically, when a synthesized pore solution is present), the presence of counter ions (Na+ and K+) and non-ionic hydrophilic OH− can lead to a reduction in swelling.

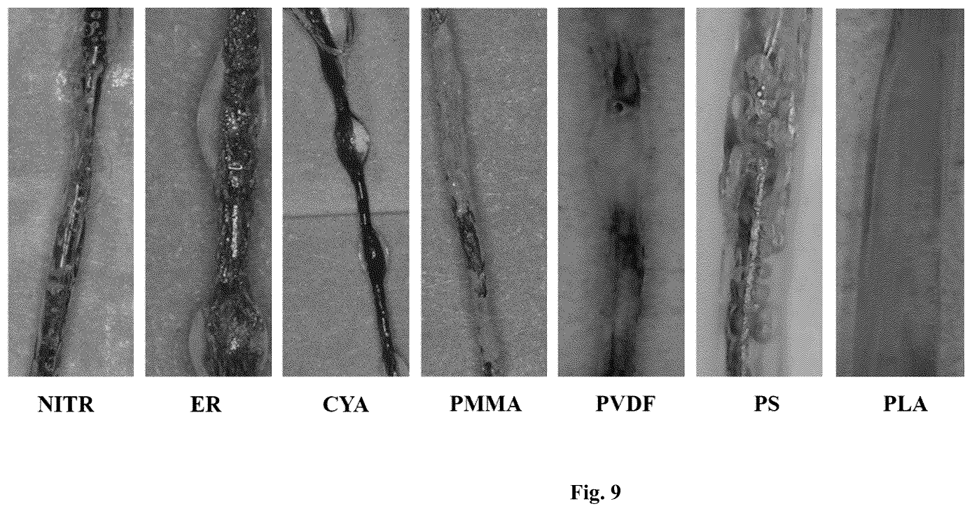

For the final fiber shell coating (on the hydrogel sheath), several polymeric materials were studied to confirm that shell materials can meet: (i) desirable coating layer morphology in terms of low coating-to-fiber ratio and uniformity, (ii) impermeability to protect endospore-laden hydrogel from undesired access to moisture/aqueous solution, (iii) and abrasion resistance during blending with the target quasi-brittle matrix. To shortlist the shell material, shell coatings with uniform coatings and a low coating-to-fiber ratio were explored. The visual observations of the BioFibers manufactured using the candidate polymers are illustrated in FIG. 9. It was observed that most of the shell material options failed in terms of coating uniformity, except for the PS and PLA. PS and PLA are both non-polar and hydrophobic polymers. PS is a brittle polymer while PLA is a relatively flexible polymer; accordingly, a blend of PS-PLA co-polymer was studied to tune flexibility/brittleness of the shell coating.

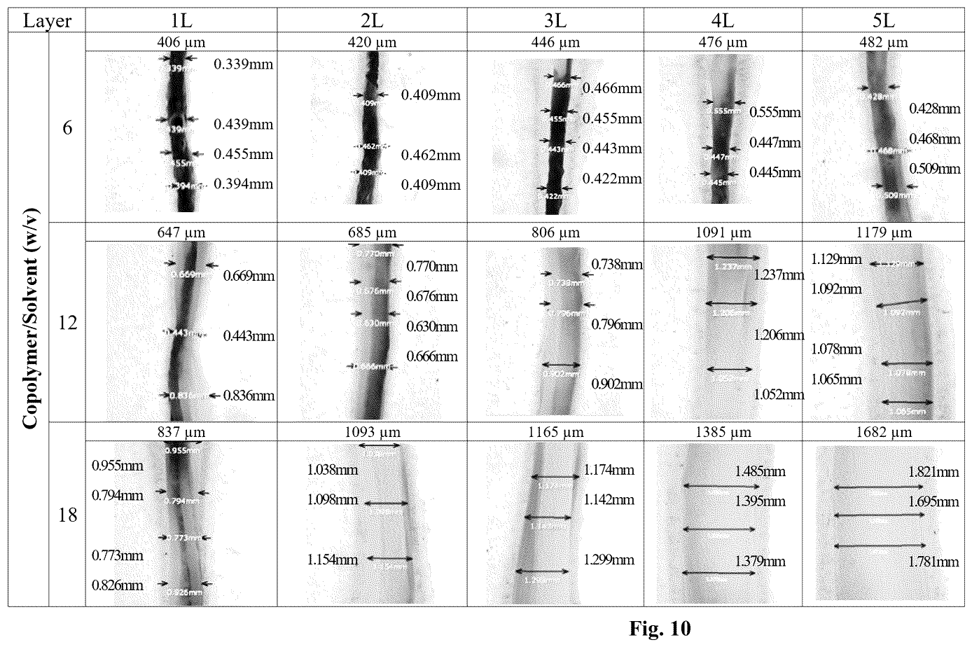

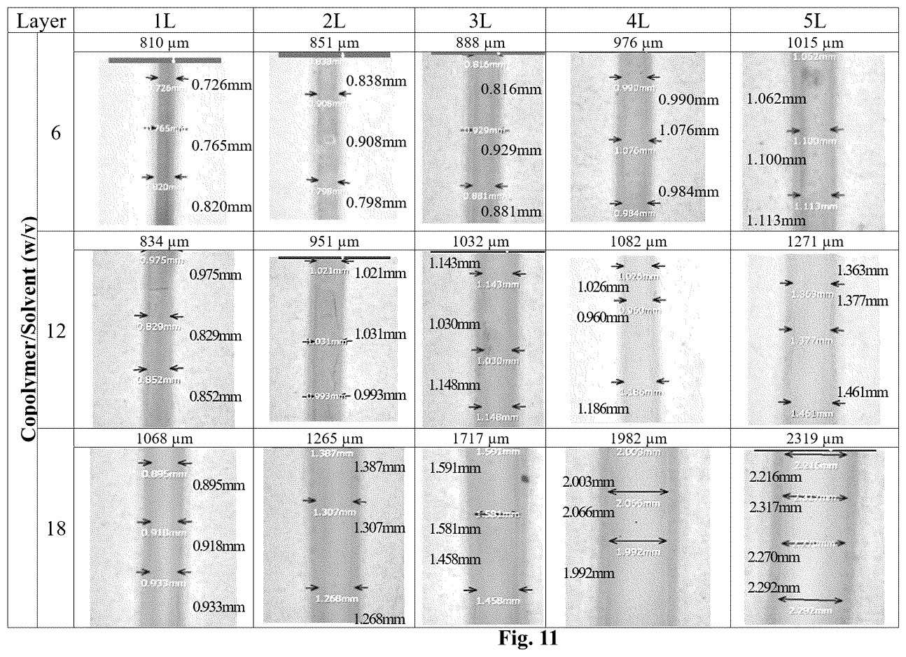

For a PS/PLA polymer blend, a mass ratio of 1:1 was selected in this study. The 1:1 wt. % polymers blend ratio was determined through an iterative process involving trial and error, whereby different values were tested and assessed to identify the optimal selection based on initial fluid ingress and abrasion survivability tests. A parametric study was conducted using 6% w/v, 12% w/v, and 18% w/v of copolymer/solvent ratio to explore the effect of copolymer solution viscosity on the shell coating thickness. Moreover, the number of shell layers applied on the core-fibers varied from 1 to 5 layers to tune the thickness and impermeability. Core-fibers coated with hydrogel crosslinked with 8 w/v Na-Alg and CA were selected and used in this section as they provided higher hydrogel coating and swelling capacity. FIG. 10 and FIG. 11 present the visual observations of the shell coating with different copolymer/solvent ratio and number of applied layers on PES and PVA core-fibers. These figures indicated that the shell coating was more uniformly adhered to PVA fiber compared to PES. This observation was mainly attributed to the rigidity of the PVA core-fiber being higher than PES core-fiber. During shell coating, the rigidity of the core-fiber was found to favor the coating process, resulting in evenly distributed shell materials on the fibers.

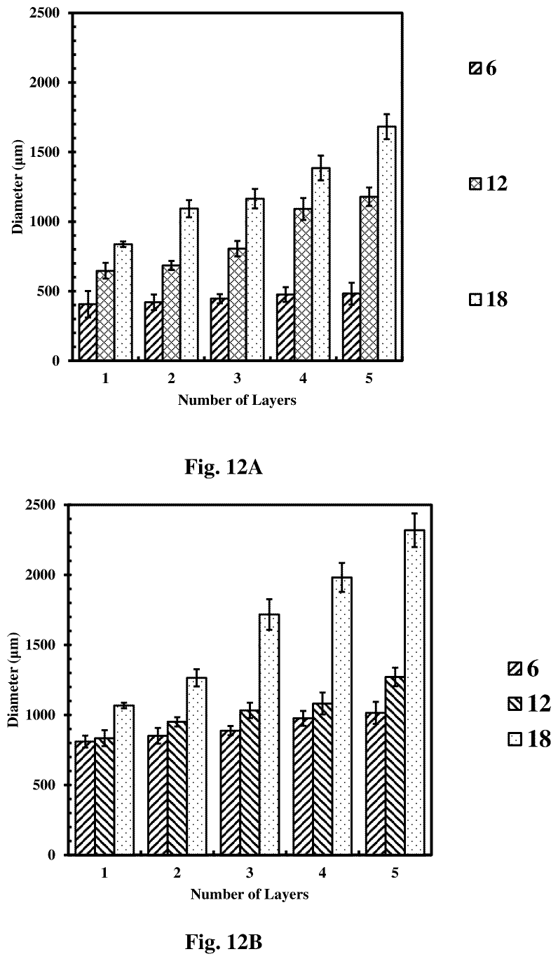

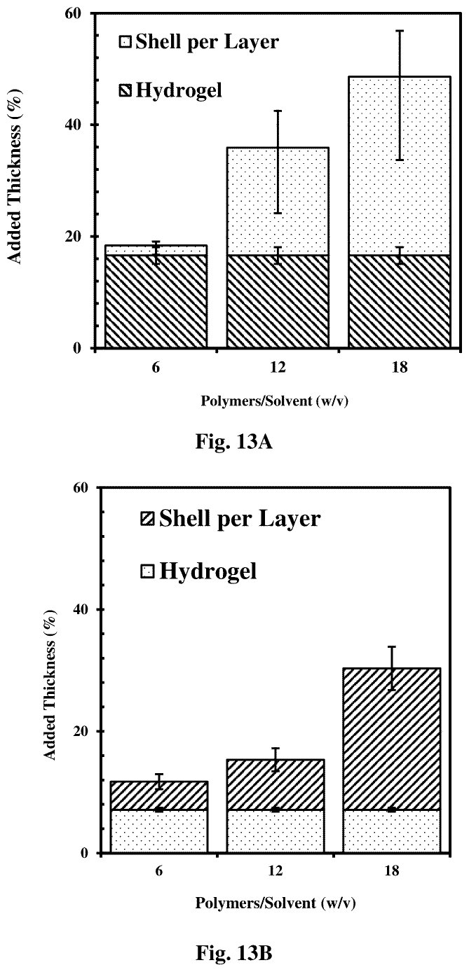

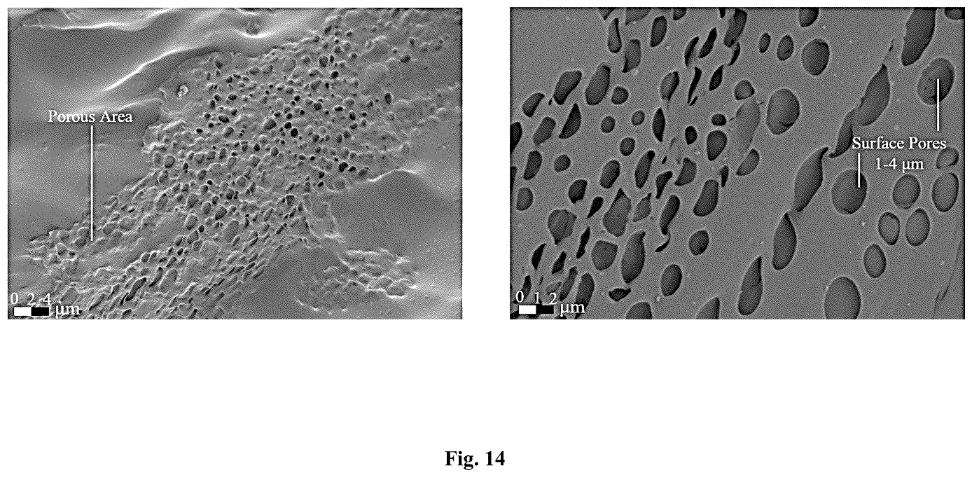

FIG. 12 shows the average and standard deviation of BioFiber diameters as copolymer/solvent and number of coating layers changed. The results revealed that a lower copolymer/solvent ratio led to a lower shell coating thickness. This was due to a lower viscosity in the shell polymer solution made using a polymer with lower molecular weights, which resulted in a lower amount of polymer adhering to the hydrogel coated core-fibers. In terms of the added thickness by the shell coating, an average of each layer of shell coating was plotted in FIG. 13. This result showed that the shell coating on PES led to higher thickness per layer, compared to PVA. For instance, the 18 w/v copolymer solution added an average of 23.20% thickness per shell layer since this value went up to 32.01% for PES. In addition, the PVA shell coating showed less thickness variation compared to PES.

### 3.4 Fluid Ingress Survivability Test

To study the shell coating thickness on the hydrogel/PVA fibers for impermeability, 1 to 5 coating layers of PLA:PS co-polymer (1:1 mass blend with 12 w/v copolymer/solvent ratio) were applied. For this section, the alginate concentration was maintained at 8 wt. % as it provided the highest swelling capacity in the hydrogel. The results for fluid ingress survivability for 1- and 2-hour(s) exposed to a basic solution (pH of ˜13) are shown in Table 3. In this table, the results are presented as pass/fail, indicating whether or not the BioFibers with the minimum diameter and shell coating survived the fluid ingress test.

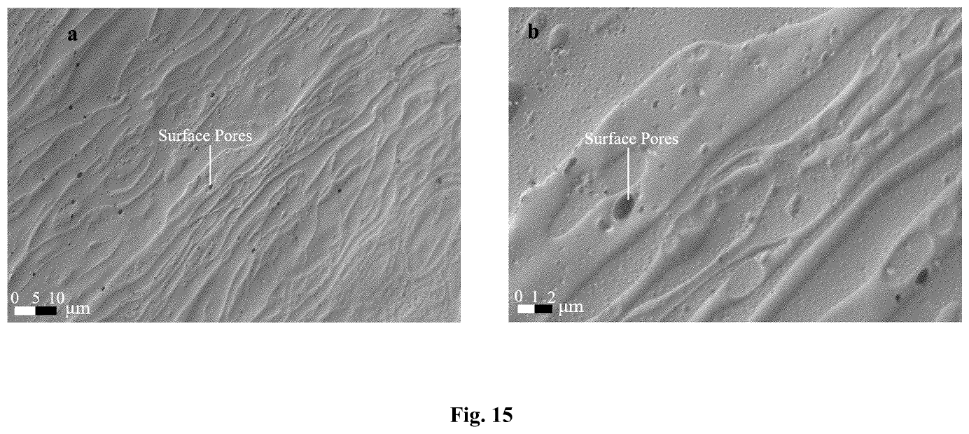

The morphology observations of PLA:PS coatings on core-fibers are shown in Figur and Error! Reference source not found. These SEM pictures indicated the pore appearance in different copolymer/solvent compositions and the number of layers. FIG. 14 indicates a one-layer coating of 6 w/v PLA:PS on the PVA; various pores with sizes in the range of 1 μm-3 μm were observed on the shell coating. The observed porous structure can be associated with solvent evaporation at ambient conditions leading to formation of three-dimensional porous films. On the other hand, the SEM pictures shown in FIG. 15 revealed that less porous structures were formed in the case of a 1-layer with a 18% w/v PLA:PS shell. An 18% w/v PLA:PS contains higher polymer and less solvent than a 6 w/v PLA:PS, and thus, more polymers can serve as building blocks leading to the formation of a less porous structure. The porous structure can adversely impact the shell survivability against the fluid ingress.

TABLE 3
Shell coating survivability against ingress of aqueous solution

| | |
|---|---|
| PES | PVA |

| w/v | Layer | 1 hr | 2 hr | 1 hr | 2 hr |
|---|---|---|---|---|---|
| 6 | 1 | F | F | F | F |
| | 2 | F | F | F | F |
| | 3 | F | F | F | F |
| | 4 | F | F | P | F |
| | 5 | F | F | P | F |
| 12 | 1 | F | F | F | F |
| | 2 | P | F | P | F |
| | 3 | P | F | P | F |
| | 4 | P | P | P | F |
| | 5 | P | P | P | P |
| 18 | 1 | P | F | P | F |
| | 2 | P | P | P | P |
| | 3 | P | P | P | P |
| | 4 | P | P | P | P |
| | 5 | P | P | P | P |

| | | | | |
|---|---|---|---|---|
| Required D_{BioFiber} (um) | 680 | 1090 | 950 | 1080 |
| Required t_{Shell} (%) | 35 | 86 | 18 | 28 |

F: Failed
P: Passed
D_{BioFiber}: Total diameter of the fiber after hydrogel and shell coatings
t_{Shell}: Shell thickness as defined in section 2.3.1

### 3.5 Shell Abrasion Resistance

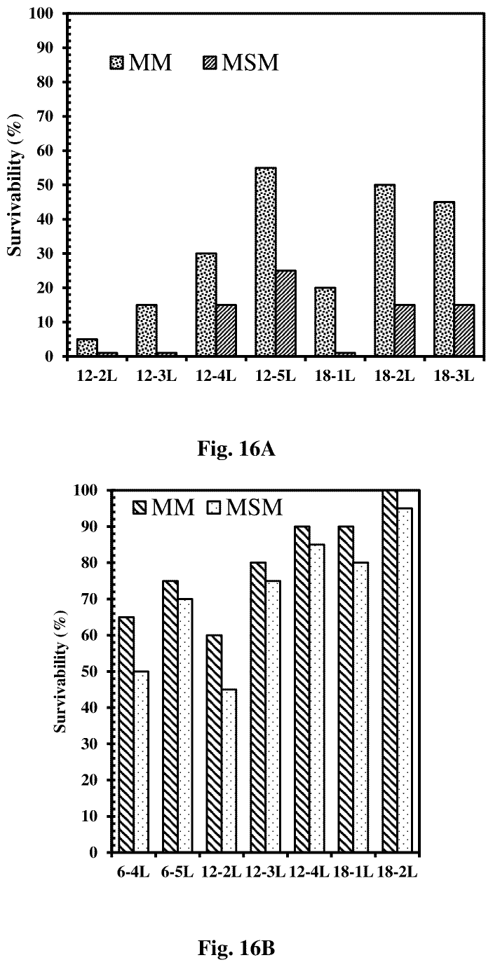

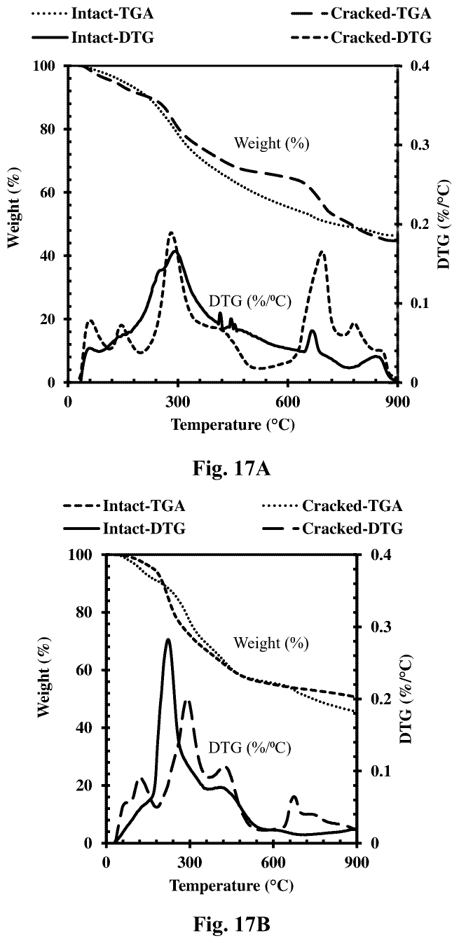

To determine the abrasion resistance of the shell coating against manual and mechanical shear mixing, the examples were subjected to the same manufacturing process, i.e., shear or manual mixing of the quasi-brittle composite. The abrasion survivability results were plotted in FIG. 16 for PES and PVA core-fibers. It was observed that a lower number of BioFibers were capable of surviving mechanical shear mixing (MSM) compared to manual mixing (MM). This was due to higher stress being applied on the shell coating in the case of mechanical mixing done by a vacuum mixer. Further, it was observed that the BioFibers made with PVA showed a higher survivability rate compared to PES. This was mainly associated with the difference in rigidity of core-fibers. The high rigidity of the core-fiber, i.e., PVA, was beneficial during the mixing stage as it resisted higher deformation. In terms of the copolymer/solvent ratio, PES with 12% w/v-5 L, and PVA with 18% w/v-2 L showed higher survivability rates.

### 3.6 MICCP Activity

Thermogravimetric analysis (TGA) was performed to quantitatively analyze the precipitation of calcium carbonate in the activated BioFibers to evaluate the self-healing capacity. BioFibers activation was achieved by delinquently cracking only shell coating using a surgical knife and microscope and then, exposing BioFibers to activation media. This activation method was applied to the BioFibers to maintain a controlled and consistent activation for all the samples. Upon exposure, the endospores were expected to be released to the media where they would be in contact with nutrients, and thus, capable of germinating, and producing carbonate ions. The carbonate ions would then be reacted with the calcium present in the media to achieve MICCP.

For the TGA test, a total of 24 tests were performed on intact and fractured BioFiber made with 8% w/v Na-Alg and PS:PLA (1:1 mass %). For the shell coating, PES coated with 12% w/v-5 L copolymer/solvent, and PVA with 18% w/v-1 L copolymer/solvent were used. After MICCP was terminated, the BioFibers were separated from solid residue, and TGA tests were conducted on residue solid mineral powder. Figur presents the weight loss curves (TGA) and derivative curves (DTG) showcasing TGA results for both intact and fractured BioFibers, providing a selection of data for analysis. In the TGA results, several weight losses were observed. The weight loss in the temperature range of 30° C.-105° C. was mainly attributed to moisture loss in the samples and the weight loss between 200° C.-600° C. was mainly attributed to organic matter decomposition into residue solid and gases [52]. The weight loss in the temperature range of 600° C.-800° C. was mainly attributed to the decomposition of calcium carbonate (CaCO₃) to calcium oxide (CaO) [53]. Calculation of the quantity of calcium carbonate was performed utilizing the following equations:

$${W}_{C a C {O}_{3}} ( \% ) = [ {WL}_{C a C {O}_{3}} \times ( {M}_{C a C {O}_{3}} / {M}_{C {O}_{2}} ) ] / ( {W}_{I n t} - {W}_{W} ) \qquad Eq . 5$$

$${W}_{C a C {O}_{3}} ( mg ) = {W}_{C a C {O}_{3}} ( \% ) \times {W}_{Res} \qquad Eq . 6$$

where WL_{CaCO3} is the weight loss between 600° C.-800° C., M_{CaCo3} and M_{CO2} are the molar weight of calcium carbonate and carbon dioxide, respectively. In order to remove the moisture in the results, the normalized weight of CaCO₃, i.e., W_{CaCO3}(%), was calculated using the initial weight of the TGA sample (W_{Int}) subtracted by the weight loss associated with the moisture loss (W_{W}). To determine the amount of calcium carbonate precipitated by each BioFiber, the weight percentage of CaCO₃ in each TGA test was multiplied by the weight of solid residue after MICCP (W_{Res}).

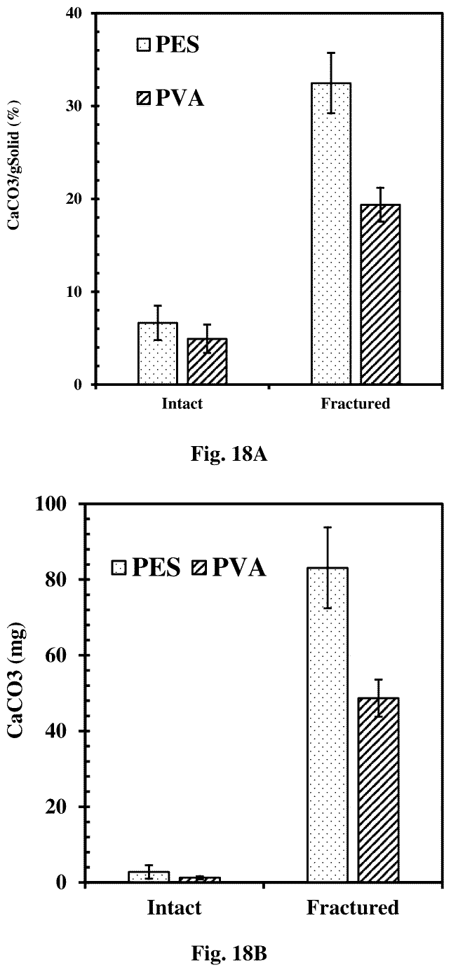

In FIG. 18, the results for the quantified amount of CaCO₃ were shown for intact and fractured BioFibers. The intact BioFibers were used as the control samples to determine the difference between precipitation in pre-activated and post-activated BioFibers. The results indicated that the fractured BioFibers produced significant calcium carbonate compared to intact samples. Although it was anticipated that the intact BioFiber would exhibit no precipitation of calcium carbonate, a small quantity was detected. This occurrence can be attributed to imperfections present at the edges of the BioFibers or the introduction of external contaminants, for example, urease-producing bacterial species, during the manufacturing process of the shell coating. These results demonstrate that the BioFibers can produce self-healing MICCP products after activation. Whereas little self-healing MICCP activity was relatively observed in intact BioFibers. The negligible to zero MICCP activity in intact BioFibers implies that the developed shell coating can protect the endospores from releasing into the media before damage occurrence, supporting the damage-responsiveness functionality of the developed BioFibers.

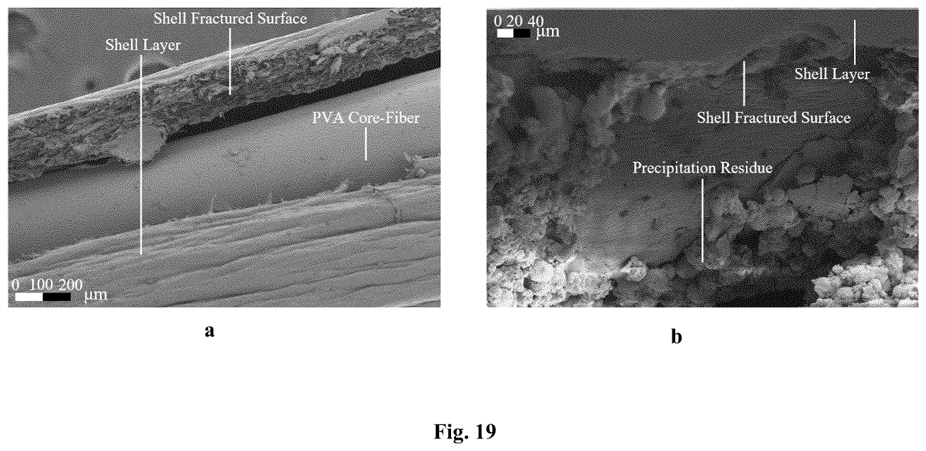

In addition to TGA results, SEM images were taken of the fractured BioFibers before and after MICCP activation. As shown in FIG. 19(a), no mineral formation was observed in BioFibers before MICCP activation. Whereas in the SEM images, shown in FIG. 19B, revealed the formation of minerals adjacent to the fractured zone after MICCP activation. The mineral formation was initiated by hydrogel swelling and endospores releasing into the media. Afterwards, the solid MICCP residues were detected on the core-fibers and the detached shell layers. These observations also acknowledge the mineral formation capacity of the developed BioFibers prior to activation.

The TGA results revealed that 32.5% and 19.4% of total precipitated solid were calcium carbonate in the PES and PVA BioFiber, respectively. The precipitations obtained only after 30 hours of BioFiber activation, which was mainly controlled by the germination phase of endospores. In terms of total weight of precipitated calcium carbonate, 83.1 mg and 48.7 mg of calcium carbonate were produced per each BioFiber with PES and PVA core-fiber, respectively.

Other embodiments of the present disclosure will be apparent to those skilled in the art from consideration of the specification and practice of the embodiments disclosed herein. As used throughout the specification and claims, “a” and/or “an” and/or “the” may refer to one or more than one. Unless otherwise indicated, all numbers expressing quantities, proportions, percentages, or other numerical values are to be understood as being modified in all instances by the term “about.” Accordingly, unless indicated to the contrary, the numerical parameters set forth in the specification and claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

It is to be understood that each component, compound, substituent or parameter disclosed herein is to be interpreted as being disclosed for use alone or in combination with one or more of each and every other component, compound, substituent or parameter disclosed herein.

It is further understood that each range disclosed herein is to be interpreted as a disclosure of each specific value within the disclosed range that has the same number of significant digits. Thus, for example, a range from 1-4 is to be interpreted as an express disclosure of the values 1, 2, 3 and 4 as well as any range of such values.

It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range. That is, it is also further understood that any range between the endpoint values within the broad range is also discussed herein. Thus, a range from 1 to 4 also means a range from 1 to 3, 1 to 2, 2 to 4, 2 to 3, and so forth.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

### REFERENCES

1. Khan, N. I. and S. Halder, Self-healing fiber-reinforced polymer composites for their potential structural applications, in Self-healing polymer-based systems. 2020, Elsevier. p. 455-472.
2. Pulikkalparambil, H., et al., Self-repairing hollow-fiber polymer composites. Self-Healing Composite Materials, 2020: p. 313-326.
3. Beaumont, P. W., et al., Comprehensive composite materials II. Vol. 6. 2018: Elsevier Amsterdam, The Netherlands.
4. Althoey, F., M. Balapour, and Y. Farnam, Reducing detrimental sulfate-based phase formation in concrete exposed to sodium chloride using supplementary cementitious materials.

Journal of Building Engineering, 2022. 45: p. 103639.
5. Laschi, C. and B. Mazzolai, Bioinspired materials and approaches for soft robotics. Mrs Bulletin, 2021. 46: p. 345-349.
6. Jogi, P. K. and T. V. Lakshmi, Self healing concrete based on different bacteria: a review.

Materials Today: Proceedings, 2021. 43: p. 1246-1252.
7. Terryn, S., et al., Toward self-healing actuators: A preliminary concept. IEEE Transactions on Robotics, 2016. 32(3): p. 736-743.
8. Ksara, M., et al., Microbial damage mitigation strategy in cementitious materials exposed to calcium chloride. Construction and Building Materials, 2019. 195: p. 1-9.
9. Zwaag, S., Self healing materials: an alternative approach to 20 centuries of materials science. Vol. 30. 2008: Springer Science+ Business Media BV Dordrecht, The Netherlands.
10. Shah, K. W. and G. F. Huseien, Biomimetic self-healing cementitious construction materials for smart buildings. Biomimetics, 2020. 5(4): p. 47.
11. Witte, M. B. and A. Barbul, General principles of wound healing. Surgical Clinics of North America, 1997. 77(3): p. 509-528.
12. Wilkinson, H. N. and M. J. Hardman, Wound healing: Cellular mechanisms and pathological outcomes. Open biology, 2020. 10(9): p. 200223.
13. He, J., C. Qiao, and Y. Farnam, Durability evaluation of reinforced concrete with surface treatment of soy methyl ester-polystyrene under freeze-thaw cycles and calcium chloride. Cement and Concrete Composites, 2023: p. 104927.
14. Althoey, F., et al., Thermo-chemo-mechanical understanding of damage development in porous cementitious materials exposed to sodium chloride under thermal cycling. Cement and Concrete Research, 2021. 147: p. 106497.
15. De Luca, I., et al., Nanotechnology development for formulating essential oils in wound dressing materials to promote the wound-healing process: A review. Applied sciences, 2021. 11(4): p. 1713.
16. Lin, P.-H., et al., Zinc in wound healing modulation. Nutrients, 2017. 10(1): p. 16.
17. Narayan, R., et al., Self-Healing: An Emerging Technology for Next-Generation Smart Batteries. Advanced Energy Materials, 2022. 12(17): p. 2102652.
18. Barbero, E. J., F. Greco, and P. Lonetti, Continuum damage-healing mechanics with application to self-healing composites. International Journal of Damage Mechanics, 2005. 14(1): p. 51-81.
19. Ekeocha, J., et al., Challenges and Opportunities of Self-Healing Polymers and Devices for Extreme and Hostile Environments. Advanced Materials, 2021. 33(33): p. 2008052.
20. Diesendruck, C. E., et al., Biomimetic self-healing. Angewandte Chemie International Edition, 2015. 54(36): p. 10428-10447.
21. Wu, D. Y., S. Meure, and D. Solomon, Self-healing polymeric materials: A review of recent developments. Progress in Polymer Science, 2008. 33(5): p. 479-522.
22. Wool, R. P., Self-healing materials: a review. Soft Matter, 2008. 4(3): p. 400-418.
23. Hager, M. D., et al., Self-healing materials. Advanced Materials, 2010. 22(47): p. 5424-5430.
24. Dinarvand, P. and A. Rashno, Review of the potential application of bacteria in self-healing and the improving properties of concrete/mortar. Journal of Sustainable Cement-Based Materials, 2022. 11(4): p. 250-271.
25. Chetty, K., et al., Self-healing bioconcrete based on non-axenic granules: A potential solution for concrete wastewater infrastructure. Journal of Water Process Engineering, 2021. 42: p. 102139.
26. Fahimizadeh, M., et al., Multifunctional, sustainable, and biological non-ureolytic self-healing systems for cement-based materials. Engineering, 2022.
27. Chuo, S. C., et al., Insights into the current trends in the utilization of bacteria for microbially induced calcium carbonate precipitation. Materials, 2020. 13(21): p. 4993.
28. Rahmaninezhad, S. A., et al., Evaluation of different strategies for efficient sporulation and germination of the MICP bacterium Lysinibacillus sphaericus strain MB284 (ATCC 13805). bioRxiv, 2022: p. 2022.09. 15.508202.
29. Riley, E. P., et al., Milestones in Bacillus subtilis sporulation research. Microbial Cell, 2021. 8(1): p. 1.
30. Souradeep, G. and H. W. Kua, Encapsulation technology and techniques in self-healing concrete. Journal of Materials in Civil Engineering, 2016. 28(12): p. 04016165.
31. Wang, J., et al., Self-healing concrete by use of microencapsulated bacterial spores. Cement and Concrete Research, 2014. 56: p. 139-152.
32. Tang, Y. and J. Xu, Application of microbial precipitation in self-healing concrete: A review on the protection strategies for bacteria. Construction and Building Materials, 2021. 306: p. 124950.
33. De Belie, N., et al., A review of self-healing concrete for damage management of structures. Advanced Materials Interfaces, 2018. 5(17): p. 1800074.
34. Sangadji, S., Can self-healing mechanism helps concrete structures sustainable? Procedia Engineering, 2017. 171: p. 238-249.
35. Wan, Z., et al., Mechanical properties and healing efficiency of 3D-printed ABS vascular based self-healing cementitious composite: Experiments and modelling. Engineering Fracture Mechanics, 2022. 267: p. 108471.
36. De Nardi, C., D. Gardner, and A. D. Jefferson, Development of 3D printed networks in self-healing concrete. Materials, 2020. 13(6): p. 1328.
37. Huseien, G. F., et al., Smart bio-agents-activated sustainable self-healing cementitious materials: an all-inclusive overview on progress, benefits and challenges. Sustainability, 2022. 14(4): p. 1980.
38. Zhu, D. Y., M. Z. Rong, and M. Q. Zhang, Self-healing polymeric materials based on microencapsulated healing agents: From design to preparation. Progress in Polymer Science, 2015. 49: p. 175-220.
39. Somasri, M. and B. N. Kumar, Graphene oxide as Nano material in high strength self-compacting concrete. Materials Today: Proceedings, 2021. 43: p. 2280-2289.
40. Zhang, W., et al., Self-healing cement concrete composites for resilient infrastructures: A review. Composites Part B: Engineering, 2020. 189: p. 107892.
41. Kim, H., et al., Recent advances in microbial viability and self-healing performance in bacterial-based cementitious materials: A review. Construction and Building Materials, 2021. 274: p. 122094.
42. Jiang, W., et al., Synthesis and self-healing properties of composite microcapsule based on sodium alginate/melamine-phenol-formaldehyde resin. Construction and Building Materials, 2021. 271: p. 121541.
43. Anglani, G., et al., Durability of self-healing cementitious systems with encapsulated polyurethane evaluated with a new pre-standard test method. Materials and Structures, 2022. 55(5): p. 143.
44. Guo, S. and S. Chidiac. Self-healing concrete: A critical review. in Proceedings of the 2019 CSCE Annual Conference, Laval, QC, Canada. 2019.
45. Sahmaran, M., G. Yildirim, and T. K. Erdem, Self-healing capability of cementitious composites incorporating different supplementary cementitious materials. Cement and Concrete Composites, 2013. 35(1): p. 89-101.
46. Li, M. and S. Fan, Designing repeatable Self-healing into cementitious materials. 2016.
47. Pang, J. W. and I. P. Bond, A hollow fibre reinforced polymer composite encompassing self-healing and enhanced damage visibility. Composites Science and Technology, 2005. 65(11-12): p. 1791-1799.
48. Araújo, M., et al., Poly (methyl methacrylate) capsules as an alternative to the “proof-of-concept” glass capsules used in self-healing concrete. Cement and Concrete Composites, 2018. 89: p. 260-271.
49. Li, V. C., Y. M. Lim, and Y.-W. Chan, Feasibility study of a passive smart self-healing cementitious composite. Composites Part B: Engineering, 1998. 29(6): p. 819-827.
50. Khaneghahi, M. H., et al. Development of bio-inspired multi-functional polymeric-based fibers (BioFiber) for advanced delivery of bacterial-based self-healing agent in concrete. in MATEC Web of Conferences. 2023. EDP Sciences.
51. Bekas, D., et al., Self-healing materials: A review of advances in materials, evaluation, characterization and monitoring techniques. Composites Part B: Engineering, 2016. 87: p. 92-119.
52. Ferral-Pérez, H., et al., Novel method to achieve crystallinity of calcite by Bacillus subtilis in coupled and non-coupled calcium-carbon sources. AMB Express, 2020. 10: p. 1-10.
53. Scrivener, K., R. Snellings, and B. Lothenbach, A practical guide to microstructural analysis of cementitious materials. Vol. 540. 2016: Crc Press Boca Raton, FL, USA.

1. A multifunctional-damage responsive biofiber comprising:
a core comprising a polymeric fiber comprising one or more polymers selected from the group consisting of polyester, polyethylene, polypropylene, polyvinyl alcohol, polyamides, aramid, polyacrylonitrile, cellulose, polyurethane, and combinations thereof;
a crosslinked endospore loaded hydrogel layer coating the core, wherein the hydrogel layer is formed with:
a solution comprising endospores and one or more anionic polymers selected from the group consisting of polysaccharides, hyaluronic acid, colominic acid, polysalic acid, chondroitin, queratane, dextrans, heparin, carrageenan, furcelerans, alginates, agar agar, glucomannan, gums, pectins, cellulose, starches, sorbitan esters, and combinations thereof, and
one or more crosslinking agents; and
a (co)polymer shell encapsulating the hydrogel layer, wherein the shell has one or more layers which may be the same or different, and each of the one or more layers is formed with a (co)polymer selected from the group consisting of nitrocellulose (NITR), epoxy Resin (ER), polymethylmethacrylate (PMMA), polyvinylidene fluoride (PVDF), cyanoacrylate adhesive (CYA), polystyrene (PS), polylactic acid (PLA), and combinations thereof.

2. The biofiber of claim 1, wherein the one or more anionic polymers comprises sodium alginate.

3. The biofiber of claim 1, wherein the one or more crosslinking agents is a cationic crosslinking agent selected from the group consisting of calcium chloride, calcium acetate, and combinations thereof.

4. The biofiber of claim 1, wherein the shell is formed from a copolymer.

5. The biofiber of claim 4, wherein the copolymer comprises a combination of two of the (co)polymers in a weight ratio of from about 0.25:1 to 1:0.25, or about 1:1.

6. The biofiber of claim 1, wherein the shell is formed from two or more layers, or three or more layers, or four or more layers, or five layers.

7. The biofiber of claim 1, wherein the hydrogel layer has a thickness of from about 0.5% to about 20% of the thickness of the core.

8. The biofiber of claim 1, wherein the polymeric fiber has a fiber length of from about 10 mm to about 100 mm, or from about 10 mm to about 70 mm, or from about 10 mm to about 60 mm.

9. The biofiber of claim 1, wherein the polymeric fiber has a ratio of a fiber length to a fiber diameter of from about 20 to 80 or from about 20 to about 60.

10. The biofiber of claim 1, wherein the hydrogel layer has a swelling ratio of from about 0 to 10, or from about 0.2 to 8, after 15 minutes of exposure to an aqueous solution, wherein the swelling ratio is determined by Equation 4:
$${S}_{r} = \frac{{W}_{s} - {W}_{d}}{{W}_{d}}$$
wherein Sᵣ is the swelling ratio, wₛ is a weight of the hydrogel layer after exposure to the aqueous solution, and wd is a weight of the hydrogel layer prior to exposure to the aqueous solution.

11. The biofiber of claim 1, wherein the hydrogel layer is configured to release the endospores upon fracture of the shell layer.

12. The biofiber of claim 1, wherein the biofiber is configured to have a function selected from the group consisting of self-healing, sensing, drug delivery, and microcrack identification in materials.

13. The biofiber of claim 1, wherein the one or more anionic polymers in the solution has a concentration of from about 1% w/v to about 20% w/v, or from about 2% w/v to about 15% w/v, based on the weight of the one or more anionic polymers in a solvent.

14. A method of forming the multi-functional damage-responsive biofiber of claim 1, comprising steps of:
a) coating the core with the solution comprising the one or more anionic polymers and endospores to form a coated core;
b) crosslinking the coated core formed in step a) with the crosslinking agent to form a crosslinked hydrogel layer coated core; and
c) encapsulating the crosslinked hydrogel layer coated core with the (co)polymer to form a polymer shell having one or more layers on the exterior in order to form the multi-functional biofiber.

15. The method of claim 14, further comprising a step of drying the crosslinked hydrogel layer coated core.

16. The method of claim 14, wherein the coating of the core in step a) is performed with a reel-to-reel system, at a speed of 10 rot/sec to 100 rot/sec or, more preferably, 50 rot/sec to 100 rot/sec.

17. The method of claim 14, wherein step a) is carried out by soaking the core in the solution, and/or wherein step b) is carried out by soaking the product of step a) in a bath comprising the crosslinking agent and crosslinking the crosslinking agent to form the crosslinked hydrogel layer coated core, and/or wherein step c) is carried out by soaking the crosslinked hydrogel layer coated core in a bath of the (co)polymer to encapsulate the crosslinked hydrogel layer coated core thereby forming the multi-functional biofiber.

18. A method for self-healing a matrix material, comprising impregnating the matrix with the multi-functional damage-responsive biofiber of claim 1 to form an impregnated matrix, wherein the multi-functional damage-responsive biofiber releases the endospore hydrogel layer upon fracture of the shell, corresponding to a fracture of the matrix to induce self-healing.

19. The method of claim 18, wherein the matrix, the core, and the shell of the multi-functional damage-responsive biofiber are defined by Equation A:
$$0.8 \leq \frac{G {G}_{s s h eeeee e}}{{GG}_{mmmmmmmmmmmm}} < 1. and \frac{G G s s h e e e e e e}{{GG}_{ccccmmee}} \leq 1.0$$
wherein Gₛₕₑₗₗ is defined as a fracture energy release rate of the polymer shell, Gₘₐₜᵣᵢₓ is defined as the fracture energy release rate of the impregnated matrix, and G_{core} is defined as the fracture energy release rate of the core.
